SEQUENCE LISTING (21 sequences)

SEQ ID NO: 1 - DNA, 427 bp, hybrid
  CDS (12)..(404)
tctagaccaccatggagtcagacacactcctgctatgggtgctgctgctctgggttccaggctccactggtgacattgtgctcacccaatctccagcttctttggctgtgtctctggggcagagagccaccatctcctgcagagccagtgaaagtgttgaatattatgtcacaagtttaatgcagtggtaccaacagaaaccaggacagccacccaaactcctcatctatgctgcatccaacgtagattctggggtccctgccaggtttagtggcagtgggtctgggacagacttcagcctcaacatccatcctgtggaggaggatgatattgcaatgtatttctgtcagcaaagtaggaaggttccattcacgttcggctcggggacaaagttggaaataaaacgtaagtagacttttgctctaga

SEQ ID NO: 2 - protein, 131 aa, hybrid
MESDTLLLWVLLLWVPGSTGDIVLTQSPASLAVSLGQRATISCRASESVEYYVTSLMQWYQQKPGQPPKLLIYAASNVDSGVPARFSGSGSGTDFSLNIHPVEEDDIAMYFCQQSRKVPFTFGSGTKLEIK

SEQ ID NO: 3 - DNA, 877 bp, hybrid
  CDS (12)..(431)
tctagaccaccatggagtcagacacactcctgctatgggtgctgctgctctgggttccaggctccactggtgacattgtgctcacccaatctccagcttctttggctgtgtctctggggcagagagccaccatctcctgcagagccagtgaaagtgttgaatattatgtcacaagtttaatgcagtggtaccaacagaaaccaggacagccacccaaactcctcatctatgctgcatccaacgtagattctggggtccctgccaggtttagtggcagtgggtctgggacagacttcagcctcaacatccatcctgtggaggaggatgatattgcaatgtatttctgtcagcaaagtaggaaggttccattcacgttcggctcggggacaaagttggaaataaaacgtaagtagacttttgctctagatctagaccaccatggctgtcctggtgctgttcctctgcctggttgcatttccaagctgtgtcctgtcccaggtgcagctgaaggagtcaggacctggcctggtggcgccctcacagagcctgtccatcacttgcactgtctctggattttcattaaccagctatggtgtacactgggttcgccagcctccaggaaagggtctggaatggctgggagtcatatggcctggtggaggcacaaattttaattcggctctcatgtccagactgagcatcagcgaagacaactccaagagccaagttttcttaaaaatgaacactctgcaaactgatgacacagccatatattattgtgccagagatcgggcgtatggtaactacctctatgccatggactactggggtcaaggaacctcagtcaccgtctcctcaggtaagaatggcctctaga

SEQ ID NO: 4 - protein, 133 aa, hybrid
MESDTLLLWVLLLWVPGSTGDIVLTQSPASLAVSLGQRATISCRASESVEYYVTSLMQWYQQKPGQPPKLLIYAASNVDSGVPARFSGSGSGTDFSLNIHPVEEDDIAMYFCQQSRKVPFTFGSGTKLEIKRK

SEQ ID NO: 5 - protein, 6 aa, hybrid
TFALDL

SEQ ID NO: 6 - DNA, 450 bp, hybrid
  CDS (12)..(431)
tctagaccaccatggctgtcctggtgctgttcctctgcctggttgcatttccaagctgtgtcctgtcccaggtgcagctgcaggagtcaggacctggcctggtgaagccctcagagaccctgtccctcacttgcgctgtctctggattttcattaaccagctatggtgtacactggattcgccagcctccaggaaagggtctggaatggctgggagtcatatggcctggtggaggcacaaattttaattcggctctcatgtccagactgaccatcagcgaagacacctccaagaaccaagtttccttaaaattgagctctgtgacagctgctgacacagccgtatattattgtgccagagatcgggcgtatggtaactacctctatgcgatggactactggggtcaaggaaccttagtcaccgtctcctcaggtaagaatggcctctaga

SEQ ID NO: 7 - protein, 140 aa, hybrid
MAVLVLFLCLVAFPSCVLSQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 8 - DNA, 427 bp, hybrid
  CDS (12)..(404)
tctagaccaccatggagtcagacacactcctgctatgggtgctgctgctctgggttccaggctccactggtgacattcagatgacccaatctccatcttctttgtctgcgtctgtgggggacagggtcaccatcacatgcagagccagtgaaagtgttgaatattatgtcacaagtttaatgcagtggtaccaacagaaaccaggaaaggcacccaaactcctcatctatgctgcatccaacgtagattctggggtcccttccaggtttagtggcagtgggtctgggacagacttcaccctcaccatctcttctctgcagccggaggatattgcaacgtattactgtcagcaaagtaggaaggttccattcacgttcggcggggggacaaaggtggaaataaaacgtaagtagacttttgctctaga

SEQ ID NO: 9 - protein, 131 aa, hybrid
MESDTLLLWVLLLWVPGSTGDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 10 - DNA, 445 bp, hybrid
  CDS (21)..(419)
tctagacagtggggaacaatatggattcacagatccaggtcctcatgtccctgctcctctggatgtctggtgcctgtggagatattgtgatgacccagtctccatattccctggctgtgtcagcaggagagaaggtcaccatgagttgcaggtccagtcagagcctctattacagtggaatcaaaaagaacctcttggcctggtaccagcagaaaccaggccagtctccgaaactgctgatctactttacatctactcggttacctggggtaccggatcgcttcacaggcagtggatctgggacagattacactctcaccatcaccagtgtccaggctgaagacatggggcattatttctgtcagcagggtataagcactccgctcacgttcggtgatggcaccaagctggagataagacgtaagtagaatccaaagtctctaga

SEQ ID NO: 11 - protein, 133 aa, hybrid
MDSQIQVLMSLLLWMSGACGDIVMTQSPYSLAVSAGEKVTMSCRSSQSLYYSGIKKNLLAWYQQKPGQSPKLLIYFTSTRLPGVPDRFTGSGSGTDYTLTITSVQAEDMGHYFCQQGISTPLTFGDGTKLEIR

SEQ ID NO: 12 - DNA, 467 bp, hybrid
  CDS (16)..(444)
tctagagtcttcaccatggtatggggcttgatcatcatcttcctggtcacagcagctacaggtgtccactcccaggtccagttgaagcagtctggggctgagcttgtgaagcctggagcctcagtgaagatatcctgcaaaacttcaggctataccttcactgatggctacatgaactgggttgagcagaagcctgggcagggccttgagtggattggaagaattgatcctgatagtggtaatactcggtacaatcagaaattccagggcaaggccacactgactagagacaaatcctccagcacagtctacatggacctcaggagcctgacatctgaggactctgctgtctattactgtgcgagagatgggaccttctacggtacctacggctactggtacttcgatttctggggccaggggacccaggtcaccgtctcctcaggtgagtccttaaaacctctaga

SEQ ID NO: 13 - protein, 143 aa, hybrid
MVWGLIIIFLVTAATGVHSQVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSS

SEQ ID NO: 14 - DNA, 46 bp, Artificial Sequence
  synthetic DNA
tatagagctcaagcttggatggtgggaagatggatacagttggtgc

SEQ ID NO: 15 - DNA, 50 bp, Artificial Sequence
  synthetic DNA
tatagagctcaagcttccagtggatagaccgatggggctgtcgttttggc

SEQ ID NO: 16 - DNA, 50 bp, Artificial Sequence
  synthetic DNA
tatagagctcaagcttccagtggatagacagatgggggtgttgttttggc

SEQ ID NO: 17 - DNA, 50 bp, Artificial Sequence
  synthetic DNA
tatagagctcaagcttccagtggatagaccgttggggctgtcgttttggc

SEQ ID NO: 18 - DNA, 50 bp, Artificial Sequence; identical to SEQ ID NO: 15
  synthetic DNA

SEQ ID NO: 19 - DNA, 50 bp, Artificial Sequence
  synthetic DNA
tatagagctcaagcttccagtggatagaccgatgggggtgttgttttggc

SEQ ID NO: 20 - DNA, 50 bp, Artificial Sequence
  synthetic DNA
tatagagctcaagcttccagtggatagtccgatggggctgtcgttttggc

SEQ ID NO: 21 - DNA, 24 bp, Artificial Sequence
  synthetic DNA
tatagagctccacttccagtgccc

(19) United States
(12) Patent Application Publication
(10) Pub. No.: US 2004/0116675 A1
(43) Pub. Date: Jun. 17, 2004

(54) Silenced anti-cd28 antibodies and use thereof

(76) Inventors: J. Jun Tso, Menlo Park, CA (US); Paul Hinton, Sunnyvale, CA (US); Maximiliano Vasquez, Palo Alto, CA (US); Kouichi Tamura, Osaka (JP); Yasuyuki Higashi, Osaka (JP); Nobuo Seki, Osaka (JP); Hirotsugu Ueda, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/450,384

(22) PCT Filed: Dec. 14, 2001
(86) PCT No.: PCT/US01/47955

(51) Int. Cl. C07K 16/44; C07H 21/04; C12N 5/06

(52) U.S. Cl. 530/387.3; 530/388.15; 536/23.53; 435/69.1; 435/328; 435/320.1

(57) ABSTRACT

The present invention provides anti-CD28 antibodies which are defective in mitogenic activity (silenced anti-CD28 antibodies), methods of producing, compositions containing the antibody and methods of immunosuppression, inducing T-cell tolerance and treating organ and/or tissue transplant rejections.

### FIELD OF THE INVENTION

This invention relates to anti-CD28 antibodies defective of mitogenic activity and to uses thereof.

### BACKGROUND OF THE INVENTION

Immune reactions, particularly organ transplant rejections, are chiefly attributed to the activation of T-lymphocytes. This activation of T cells is induced by a signal from antigen-presenting cells (APC). The signal from the APC involves a first signal via the T-cell receptor (TCR) and a second signal (costimulatory signal) via costimulatory molecules. The first signal is from the major histocompatibility antigen (MHC) complex of peptides antigen where the APC is presents the T-cell antigen through the TCR The second signal is mediated by several co-stimulatory molecules, examples of which include B7 (B7-1 (CD80) and B7-2 (CD86)) are known as ligands on the APC side and CD28, CTLA-4, etc. as receptors on the T-cell side. The ligand B7 is a glycoprotein belonging to the immunoglobulin super family and is expressed in B cells etc. which belong to the antigen-presenting cell group. Both CD28 and CTLA-4, which recognize B7 as the common ligand, are transmembrane glycoproteins belonging to the immunoglobulin super family. Thus, the activation of T cells is regulated by the concurrent transduction of the first signal via TCR and the second signal from, e.g., the B7 and CD28/CTLA-4. The signal from B7-to CD28 is known to promote whereas the signal from B7 to CTLA-4 inhibits the activation of T cells [Waterhouse et al., Science, 270:985-988 (1995)].

Heretofore, for the purpose of inducing immunosuppression or tolerance, attempts have been made to block the B7-CD28 signal by administering CTLA-4Ig, anti-B7-1 antbiody/anti-B7-2 antibody, anti-CD28 antibody or the like. For example, CTLA-4Ig binds to B7 thereby interfering with the reaction between B7 and CD28 and, as a consequence, block the signal from CD28 to exhibit immunosuppressive activity. However, since the reaction between B7 and CTLA-4 is also inhibited simultaneously, the signal of CTLA-4 acting negatively on the activation of T cells is also suppressed so that the desired tolerance is not induced (Kirk et al., Proc. Natl. Acad. Sci. USA, 94:8789-8794 (1997). An anti-B7 antibody was also prepared and reported to have suppressed activation of T cells but just as in the case of CTLA4Ig, it suppressed the CTLA-4 signal as well. An anti-CD28 antibody, in an in vitro experiment, was found to produce a mitogenic effect on T cells, and the combination of the stimulation with this antibody and an anti-CD3 antibody promoted the growth and activation of T cells and enhanced the production of cytokines [WO 90/05541, Eur. J. Immunology, 16, 1289-1296 (1986), etc.]. Furthermore, mitogenic stimulation of the CD28 receptor of the T cell by an anti-CD28 antibody has been stimulated in vivo resulted in the generation of a T-cell activation signal similar to the second signal from B7 to CD28 [Yin et al., J. Immunology, 163:4328-4334 (1999)]. These T-cell activating functions suggested that an anti-CD28 antibody might be used as an immunopotentiator in the therapy of cancer and AIDS (WO 90/05541).

### SUMMARY OF THE INVENTION

The anti-CD28 antibodies prepared by conventional technologies exert a mitogenic action on T cells. Although the reasons for this mitogenic activity is not fully understood, the binding of the anti-CD28 Fc region to the Fc receptor of the antigen-presenting cell is believed to be the probable reason (Cole et al., J. Immunology, 36:159 (1997). Therefore, by utilizing genetic engineering technology, we introduced mutations into the Fc receptor binding site of the anti-CD28 antibody to modify the antibody so that it would no longer have mitogenic activity. One such antibody the present inventors have generated, TN228 IgG2M3, in which IgG2M3 has two amino acid substitution in IgG gene. Furthermore, we demonstrated that the resulting silenced anti-CD28 antibody has no mitogenic activity which is very usefull for inducing T cell tolerance.

Therefore, the present invention provides anti-CD28 antibodies having no mitogenic activity (hereinafter referred to as silenced anti-CD28 antibodies), and a methods of suppressing immune reactions, particularly transplant rejections, and inducing immunotolerance by using said antibodies.

An object of the present invention is a silenced anti-CD28 antibody, where the anti-CD28 antibody may be a chimeric antibody and/or a humanized antibody. The variable regions of the anti 28 antibodies may include the amino acid sequences shown in SEQ ID NOS: 2, 4, 6 and 8 and polynucleotides encoding such amino acid sequences. For example, such polynucleotides include SEQ ID NOS: 1, 3, 5, and 7.

Another object of the present invention is vectors and cell hosts comprising the polynucleotides which encode the anti-CD28 antibodies.

Another object of the present invention is methods for producing the silenced anti-CD28 antibody by culturing a cell host comprising the polynucleotides which encode the anti-CD28 antibodies under conditions which allow expression of the polynucleotide and collecting the gene products produced.

Another object of the present invention is a pharmaceutical composition comprising one or more of the silenced anti-CD28 antibodies, preferably admixed with one or more pharmaceutically acceptable ingredients.

The silenced anti-CD28 antibodies are useful for inducing T-cell tolerance, immunosuppression and as a prophylactic/therapeutic drug for organ or tissue transplant rejection. Accordingly, the present invention provides methods for inducing T-cell tolerance, immunosuppression, and providing a prophylaxis or treatment therapy during an organ or tissue transplant rejection by administering one or more of the silenced anti-CD28 antibodies to a mammal. Preferably, such silenced anti-CD28 antibodies are administered in as a pharmaceutical composition as described herein and may include additional drug/pharmaceuticals where appropriate.

### BRIEF DESCRIPTION OF DRAWINGS

FIG. 1. Plasmid constructs for ChTN228 antibody expression. VL and VH of murine TN228 were constructed as mini-exons flanked by Xbal sites. The VL sequence was incorporated into the expression vector pVk and the VH sequence was incorporated into the expression vector pVg2M3.

FIG. 2. Nucleotide sequences and deduced amino acid sequences of the light chain of ChTN228 in the mini-exons. The signal peptide sequences are in italics. The CDRs are underlined. The mature light chain begins with an aspartic acid residue (bold letter). Untranslated and intron sequences are in lower case. (SEQ ID NOS: 1 and 2).

FIG. 3. Nucleotide sequences and deduced amino acid sequences of the heavy chain variable regions of ChTN228 in the mini-exons. The signal peptide sequences are in italics. The CDRs are underlined. The mature heavy chain begins with a glutamine residue (bold letter). Untranslated and intron sequences are in lower case. (SEQ ID NOS: 3 and 4).

FIG. 4. Competition experiment. P815/CD28⁺ cells were incubated with 25 ng of MuTN228-FITC and two-fold serial dilutions of either ChTN228 or MuTN228 as described. P815/CD28′ cells were also incubated with MuTN228-FITC alone, without any competitor. The mean channel fluorescence for each sample was plotted against the concentration of competitor.

FIG. 5. Inhibition effect of TN228-IgG2m3 on human primary MLR(1). Percentage inhibition of primary MLR from four individuals were shown separately.

FIG. 6. Inhibition effect of TN228-IgG2m3 on human primary ML(2). Percentage inhibition of primary MLR from four individuals were shown separately.

FIG. 7. The effect of TN228-IgG2m3 on secondary MLR

The data from two volunteers were shown separately.

[³II]-thymidine uptake in 2nd MLR were presented as percentage of dpm of Raji stimulation alone in 1st MLR as 100. TN228-IgG2m3:0.1 ug/mL

FIG. 8. Plasmid constructs for HuTN228 antibody expression. VL and VH of humanized TN228 were constructed as mini-exons flanked by XbaI sites. The VL sequence was incorporated into the expression vector pVk and the VH sequence was incorporated into the expression vector pVg2M3

FIG. 9. Nucleotide sequences and deduced amino acid sequences of the heavy chain variable regions of HuTN228 in the mini exons. The signal peptide sequences are in italics. The CDRs are underlined. The mature heavy chain begins with a glutamine residue (bold letter). (SEQ ID NOS: 5 and 6)

FIG. 10. Nucleotide sequences and deduced amino acid sequences of the light chain variable regions of HuTN228 in the mini exons. The signal peptide sequences are in italics. The CDRs are underlined. The mature light chain begins with an aspartic acid residue (bold letter). (SEQ ID NOS:7 and 8)

FIG. 11. FACS competition assay. The binding of FITC-labeled MuTN228 to P815/CD28′ cells in the presence of various amounts of competitor MuTN228 or HuTN228 antibody was analyzed in a flow cytometry competition experiment as described in the examples.

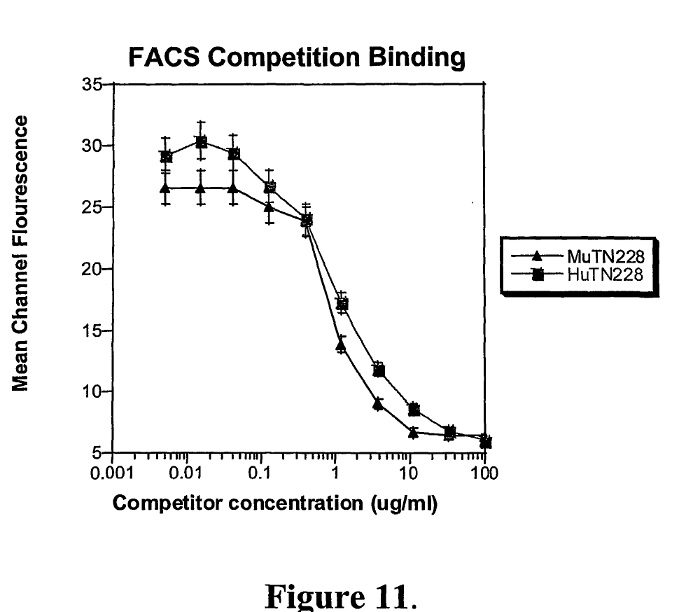

FIG. 12. ELISA competition assay. The binding of biotinylated MuTN228 to sCD28-Fc in the presence of various amounts of competitor MuTN228 or HuTN228 antibody was analyzed in an ELISA competition experiment as described in the examples.

FIG. 13. I-125 competition assay. The binding of ¹²⁵I labeled MuTN228 to P815/CD28⁺ cells in the presence of various amounts of competitor MuTN228 or HuTN228 antibody was analyzed in an ¹²⁵I labeled antibody competition experiment as described in the examples.

FIG. 14. Plasmid constructs for PV1-IgG3 antibody expression. V_{L} and V_{H} of PV1 were constructed as mini-exons flanked by XbaI sites. The V_{L} sequence was incorporated into the expression vector pMVk.rg.dE, and the V_{H} sequence into the expression vector pMVg3.D.Tt. The two plasmids were then recombined to generate a single plasmid co-expressing the heavy and light chains of PV1-IgG3.

FIG. 15A. Sequences of cDNA and deduced amino acid sequences of the light chain and heavy chain in the mini-exons. The CDRs are underlined. The mature light chain begins with an aspartic acid residue (double underlined) at position 20. (SEQ ID NOS:9 and 10).

FIG. 15B. Sequences of cDNA and deduced amino acid sequences of variable regions of PV1 in the mini-exons. The CDRs are underlined. The mature heavy chain with glutamine (double underlined) at position 20. (SEQ ID NOS: 11 and 12).

FIG. 16. Analysis of PV-1-IgG3 by size exclusion chromatography using HPLC as described in Methods. The protein was monitored by its absorbance at 280 nM.

FIG. 17. SDS-PAGE analysis of mouse IgG3 isotype control (lane 1), PV1 (lane 2), and PV1-IgG3 (lane 3). Proteins in Panel A were run under nonreducing conditions, and in Panel B reducing conditions. MW represents molecular weight markers. The numbers are MW standards in kD.

FIG. 18. EL4 cells stained with PV1 (A), 37.51 (B), or PV1-IgG3 (C), and analyzed by flow cytometry. Secondary antibodies used were: FITC-conjugated donkey anti-Armenian hamster IgG (H+L) for PV1, FITC-conjugated donkey anti-Syrian hamster IgG for 37.51, and FITC-conjugated goat anti-mouse kappa for PV1-IgG3. The solid line profiles represent cells stained with secondary antibodies only. The broken line profiles represent cells stained with both primary and secondary antibodies as described in Methods, Mouse IgG3 isotype control did not stain EL4 cells (data not shown).

FIG. 19. (A). Excess PV1, or PVI-IgG3 competes with R-PE-conjugated PV1 for binding to EL4 cells. Thin solid line (black) in flow cytometry histogram represents cells without any staining, thick solid line (dark blue) cells stained with R-PE-PV1 alone, thin broken line (magenta) cells stained with R-PE-PV1 and excess unconjugated PV1, and thin double broken line (light blue) cells stained with R-PE-PV1 and excess unconjugated PV1-IgG3. Excess mouse IgG3 isotype control had no effect on R-PE-PV1's binding to ETA cells (data not shown). (B). Excess 145.2C11, or 145.2C11-IgG3 compete with R-PE-conjugated 145.2C11 for binding to EL4. Thin solid line (black) represents cells without any staining, thick solid line (dark blue) cells stained with R-PE-145.2C11 alone, thin broken line (magenta) cells stained with R-PE-145.2C11 and excess unconjugated 145.2C11, and thin double broken line (light blue) cells stained with R-PE-145.2C11 and excess unconjugated 145.2C11-IgG3. (C). Excess PV1 competes with PV1-IgG3 for binding to EL4 cells. EL4 cells were stained with PV1-IgG3 with or without excess PV1. Cells were washed and stained with mouse IgG3-specific, FITC-conjugated donkey anti-mouse IgG (H+L). Thin solid line (black) represents cells stained with secondary antibodies only, thick solid line (dark blue) cells stained with PV1-IgG3 and secondary antibodies, and thin broken line (magenta) cells stained with PV1-IgG3 and excess PV1, and secondary antibodies.

FIG. 20. Mouse splenic cells stained with PV1-IgG3 and 145.2C11. Cells were stained with mouse IgG3 isotype control (A) or PV1-IgG3 (B), counter-stained with R-PE-conjugated goat anti-mouse IgG3 and with FITC-conjugated 145.2C11, and analyzed by two-color flow cytometry as described in Materials and Methods. Only cells in the lymphocyte gate were analyzed. PV1-IgG3-positive cells are in the upper quadrants and CD3-positive cells are in the right side quadrants. The number in each quadrant represents percentage of the cells in that particular quadrant

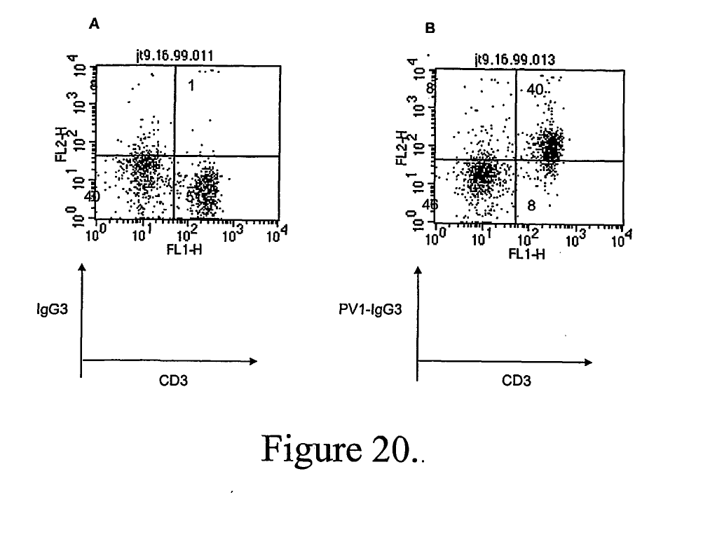

### DETAILED DESCRIPTION OF THE INVENTION

In the context of this invention, the term “silenced anti-CD28 antibody” means any anti-CD28 antibody defective of mitogenic activity. More specifically, it is an antibody which binds specifically to the antigen CD28 receptor on the surface of the T cell and does not promote the growth or activation of T cells by combined stimulation with an anti-CD3 antibody.

A silenced anti-CD28 antibody can be constructed on the basis of an anti-CD28 antibody or an anti-CD28 antibody-producing hybridoma by mutating or modifying an agonistic anti-CD28 antibody by a genetic engineering technique or by chemical modification. Taking the use of genetic engineering technology as an example, the binding affinity of the anti-CD28 antibody for the Fc receptors can be reduced or eliminated by introducing a mutation into the amino acid sequence of the Fc domain of the antibody. For example, a silenced anti-CD28 antibody can be obtained by isolating cDNA from hybridoma cells capable of producing an anti-CD28 monoclonal antibody and introducing a mutation(s) into the region of the sequence corresponding to the Fc domain which plays an important role in the binding to the Fc receptor (WO 88/07089). The site of mutation is not particularly restricted inasmuch as the binding to the Fc receptors may be inhibited. Thus, in the case of a Class IgG antibody, for instance, the H-chain amino acid residues 234, 235, 236, 237, 318, 320 and 322 are preferred and a silenced anti-CD28 antibody can be constructed by replacing at least one of these amino acids with a different amino acid.

The source of such a silenced anti-CD28 antibody can be judiciously selected according to the target animal in which the antibody is used. For example, nonhuman monoclonal antibodies contain amino acid sequences showing antigenicity in humans over a fairly broad range. Many studies have shown that the immune response of a patient to a foreign antibody following injection of the antibody is remarkably intense and the very administration of the antibody may bring the patient into a perilous condition or deprive the antibody of the therapeutic utility. Therefore, it is recommendable to replace the Fc region so as to make the antibody relatively more homologous to the therapeutic target animal, replace the framework potions of the variable regions, or use the antibody obtained from a transgenic animal into which the antibody gene of the target anima has been introduced. For example, when the antibody is to be administered to a human, a chimeric antibody (EP125023) available on replacement of the Fe region, a humanized antibody with the framework portion replaced (EP0239400, EP045126) or a human antibody (EP546073, WO 97/07671) obtained from a transgenic animal into which the human antibody gene has been introduced. By introducing mutations in these antibodies by genetic engineering techniques such as those described above or by chemical modification, the mitogenic activity of the antibodies can be reduced or eliminated.

As specific examples of the anti-CD28 antibody having a silenced Fc region, there can be mentioned not only the antibodies described hereinafter in the Examples section but also the antibodies synthetically prepared using the constant region gene of the therapeutic target animal and the variable region polynucleotides based on the amino acid sequences of variable regions shown in SEQ ID NO:2 and NO:4 or SEQ ID NO:6 and NO:8. Examples of such polynucleotides are SEQ ID NOS:1, 3, 5, and 7.

More specific examples of this invention are HuTN228 and MuTN228 and Fab fragments thereof F(ab)′2 fragments thereof, derivatives thereof and etc.

As appreciated by those skilled in the art, because of third base degeneracy, almost every amino acid can be represented by more than one triplet codon in a coding nucleotide sequence. Further, minor base pair changes may result in variation (conservative substitution) in the amino acid sequence encoded, are not expected to substantially alter the biological activity of the gene product. Thus, a nucleic acid sequencing encoding a protein or peptide as disclosed herein, may be modified slightly in sequence (e.g., substitution of a nucleotide in a triplet codon), and yet still encode its respective gene product of the same amino acid sequence.

The term “expression vector” refers to an polynucleotide which encodes the peptide of the invention and provides the sequences necessary for its expression in the selected host cell. Expression vectors will generally include a transcriptional promoter and terminator, or will provide for incorporation adjacent to an endogenous promoter. Expression vectors will usually be plasmids, further comprising an origin of replication and one or more selectable markers. However, expression vectors may alternatively be viral recombinants designed to infect the host, or integrating vectors designed to integrate at a preferred site within the host's genome. Examples of expression vectors are disclosed in Molecular Cloning: A Laboratory Manual, Second Edition, Sambrook, Fritsch, and Maniatis, Cold Spring Harbor Laboratory Press, 1989.

Suitable host cells for expression of the silenced anti-CD28 antibody include prokaryotes, yeast, archae, and other eukaryotic cells. Appropriate cloning and expression vectors for use with bacterial, fungal, yeast, and mammalian cellular hosts are well known in the art e.g., Pouwels et al. Cloning Vectors: A Laboratory Manual, Elsevier, N.Y. (1985). Preferably, the cells are mammalian cells. The vector may be a plasmid vector, a single or double-stranded phage vector, or a single or double-stranded RNA or DNA viral vector. Such vectors may be introduced into cells as polynucleotides, preferably DNA, by well known techniques for introducing DNA and RNA into cells. The vectors, in the case of phage and viral vectors also S may be and preferably are introduced into cells as packaged or encapsulated virus by well known techniques for infection and transduction. Viral vectors may be replication competent or replication defective. In the latter case viral propagation generally will occur only in complementing host cells. Cell-free translation systems could also be employed to produce the proteins using RNAs derived from the present DNA constructs.

The silenced anti-CD28 antibodys/proteins can be purified by isolation/purification methods for proteins generally known in the field of protein chemistry. More particularly, there can be mentioned, for example, extraction, recrystallization, salting out with ammonium sulfate, sodium sulfate, etc., centrifugation, dialysis, ultrafiltration, adsorption chromatography, ion exchange chromatography, hydrophobic chromatography, normal phase chromatography, reversed-phase chromatography, gel filtration method, gel permeation chromatography, affinity chromatography, electrophoresis, countercurrent distribution, etc. and combinations of these.

According to the present invention, purified antibodies may be produced by the recombinant expression systems described above. The method comprises culturing a host cell transformed with an expression vector comprising a DNA sequence that encodes the protein under conditions sufficient to promote expression of the protein. The protein is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium.

The silenced anti-CD28 antibody when formulated into a pharmaceutical composition can be used in (a) transplant rejections following the transplantation of organs or tissues, such as heart, kidney, liver, bone marrow, skin, cornea, lung, pancreas, small intestine, muscle, nerve, etc.; (b) graft-versus-host reactions in the transplantation of bone marrow; (c) autoimmune diseases such as rheumatoid arthritis, systemic lupus erythematosus, multiple sclerosis, myasthenia gravis, type I diabetes, etc.; and (d) immune diseases such as asthma, atopic dermatitis, etc.

While the silenced anti-CD28 antibody by itself can be expected to suppress immune reactions and transplant rejections and induce immunotolerance, it can also be used in combination with other drugs. Among such other drugs which are useful for combining with the silence anti-CD28 antibody are various immunosuppressants such as rapamycin, deoxyspergaulin, anti-CD40 antibody, anti-CD40L antibody, prograf, cyclosporin A, anti-IL-2 antibody, anti-IL-2 receptor antibody, anti-IL-12 antibody, anti-IL12 receptor antibody and MMF. Rapamycin, in particular, inhibits transduction of the signal related to growth of T cells among signals from the IL2 receptor but does not inhibit transduction of the apoptosis-related signal, so that its use in combination with a specific inhibitor of the CD28 signal is expected to be useful.

The silenced anti-CD28 antibody of this invention can be administered orally or parenterally, preferably by the intravenous, intramuscular or subcutaneous route.

The silenced anti-CD28 antibody of this invention can be prepared in the form of a solution or a lyophilized powder and, where necessary, may be formulated with various pharmaceutically acceptable additives such as an excipient, diluent, stabilizer, isotonizing agent and buffer. The preferred additives include a sugar such as maltose, a surfactant such as polysorbate, an amino acid such as glycine, a protein such as human serum albumin, and a salt such as sodium chloride.

Also, the dosage form such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like can be selected appropriately depending on the administration method, and the peptide of the present invention can be accordingly formulated. Formulation in general is described in Chapter 25.2 of Comprehensive Medicinal Chemistry, Volume 5, Editor Hansch et al, Pergamon Press 1990.

The dosage of the pharmaceutical composition of this invention is dependent on the specific composition, the type of disease as the target of therapy or prophylaxis, the method of administration, the patient's age and condition and the duration of treatment, among other variables. However, in the case of intravenous, intramuscular or subcutaneous administration, 0.01-100 mg/kg preferably 0.1-10 mg/kg, per day per adult can be administered.

When the silenced anti-CD28 antibody of this invention is used for suppression of transplant rejection or induction of immunotolerance in an organ or tissue transplantation, the composition can be administered in a dose of about 1 mg/kg/day immediately before transplantation, immediately after transplantation, and 3, 7, 12, 18, 25, 35, 45 and 60 days after transplantation, by intravenous, intramuscular or subcutaneous injection. The administration frequency and dosage may be judiciously increased or decreased while the course of rejection reaction after transplantation is monitored.

While the administration interval depends on the method of administration used and the patient's condition, among other factors, not only continuous administration but also intermittent administration is feasible. Thus, since the silenced anti-CD28 antibody of this invention is an antibody, it provides a sustained effect so that intermittent dosing may be rewarded with the expected efficacy. As to the period of treatment, once a tolerant state is established, this tolerance can be maintained even if the use of the silenced anti4M28 antibody is discontinued. In this respect, this silenced anti-CD28 antibody is undoubtedly superior to other immunosuppressants the immunosuppressive effect of which declines after discontinuation.

### EXAMPLES

Having generally described this invention, a further understanding can be obtained by reference to certain specific examples which are provided herein for purposes of illustration only, and are not intended to be limiting unless otherwise specified. The examples below are carried out using standard techniques, that are well known and routine to those of skill in the art, except where otherwise described in detail.

### Example 1

### Amino Acid Sequencing of the Mouse Anti-human CD28 Antibody

The hybridoma producing anti-human CD28 antibody (clone:TN228, mouse IgG1 kappa) was generously provided by Dr. Yagita (Juntendo University School of Medicine, Japan). Approximately 0.2 mg of purified anti-human CD28 antibody (TN228) was reduced in 0.64 M guanidine-HCI, 0.28 M Tris-HCI, pH 8.5, 0.055 M DT7 for 90′ at 60 C (under argon), carboxymethylated by addition of iodoacetic acid to 0.13 M for 45′ at room temperature (in the dark), followed by addition of DTr to 0.32 M (to terminate the carboxymethylation reaction), and immediately buffer-exchanged in 0.1 M sodium phosphate, 0.002 M EDTA, pH 8.0 using a PD-10 column (catalog #17-0851-01, Amersham Pharmacia Biotech, Uppsala, Sweden). The eluate was adjusted to 0.005 M DTT, 0.02% glycerol, and one third of the solution (about 0.35 ml) was transferred to a separate tube for N-terminal deblocking of the heavy chain. The sample was digested with 1800 μU of pyroglutamate aminopeptidase (catalog # 7334, Takara Shuzo Co., Ltd., Tokyo, Japan) for 24 hours at 45 C. The N-terminal sequences of the light and heavy chains from the deblocked sample were determined by 20 cycles of automated Edman degradation and PTH analysis-on a Model 241 Protein Sequencer (Hewlett Packard, Palo Alto, Calif.). The PTH derivatives were analyzed on a Hypersil ODS C18 column. The sequencer and HPLC were operated according to the manufacturer's instructions using reagents, solvents, and columns obtained from Hewlett Packard.

N-terminal sequencing results for TN228 deblocked with pyroglutamate aminopeptidase were as follows:

| residue | amino |
|---|---|
| no. | acid |
| 1 | D, Q |
| 2 | l, v |
| 3 | V, Q |
| 4 | L |
| 5 | T, K |
| 6 | Q, E |
| 7 | s |
| 8 | P, G |
| 9 | A, P |
| 10 | S, G |
| 11 | L |
| 12 | A, V |
| 13 | V, A |
| 14 | S, P |
| 15 | L, S |
| 16 | G, Q |
| 17 | Q, S |
| 18 | R, L |
| 19 | A, S |
| 20 | T, I |

### Example 2

### Cloning of Variable Region cDNAs

The V region cDNAs for the light and heavy chains of TN228 were cloned from the hybridoma cells by an anchored polymerase chain reaction (PCR) method described by Co et al. (Co, M. S., N. M. Avadalovic, P. C. Caron, M. V. Avadalovic, D. A. Scheinberg, and C. Queen. 1992. Chineric and humanized antibodies with specificity for the CD33 antigen. J. Immunol. 148: 1149-1154.). Amplification was performed on cDNA using 3′ primers that anneal respectively to the mouse kappa and gamma chain C regions, and a 5′ primer that anneals to the added G-tail of the cDNA. For VL PCR, the 3′ primer has the sequence (SEQ ID NO:13):

5′ TATAGAGCTCAAGCTTGGATGGTGGGAAGATGGATACAGTTGGTGC 3′

with residues 17-46 hybridizing to the mouse Cₖ region. For VH PCR, the 3′ primers have the degenerate sequences (SEQ ID NOS:14, 15 and 16):

```
                                                                A G T
5′ TATAGAGCTCAAGCTTCCAGTGGATAGACCGATGGGGCTGTCGTTTTGGC 3′
                                                                    T
```

with residues 17-50 hybridizing to mouse IgG C_{H}1. The non-hybridizing sequences in the two primer sets contain restriction sites used for cloning. The VL and VH cDNAs were subcloned into a TOPOII Blunt vector (Invitrogen, Inc., Carlsbad, Calif.) for sequence determination.

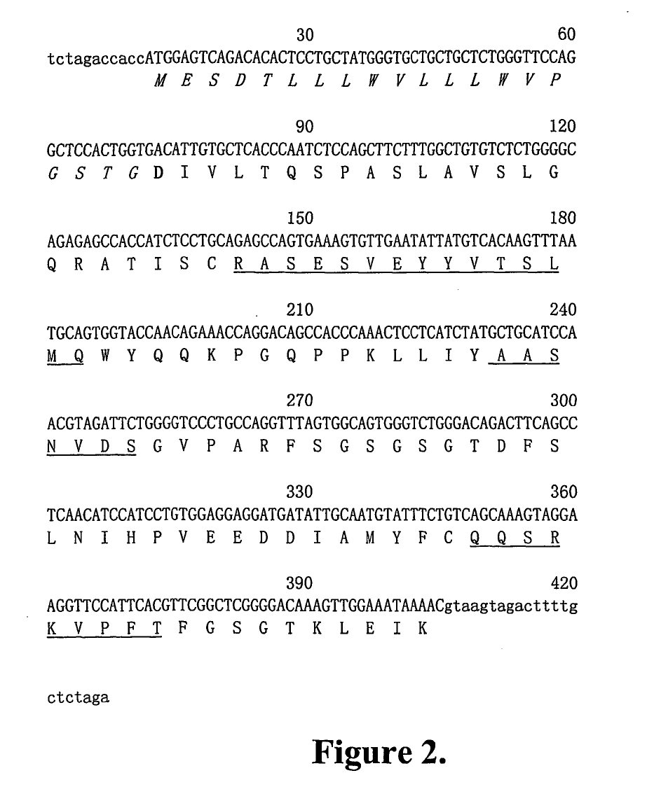

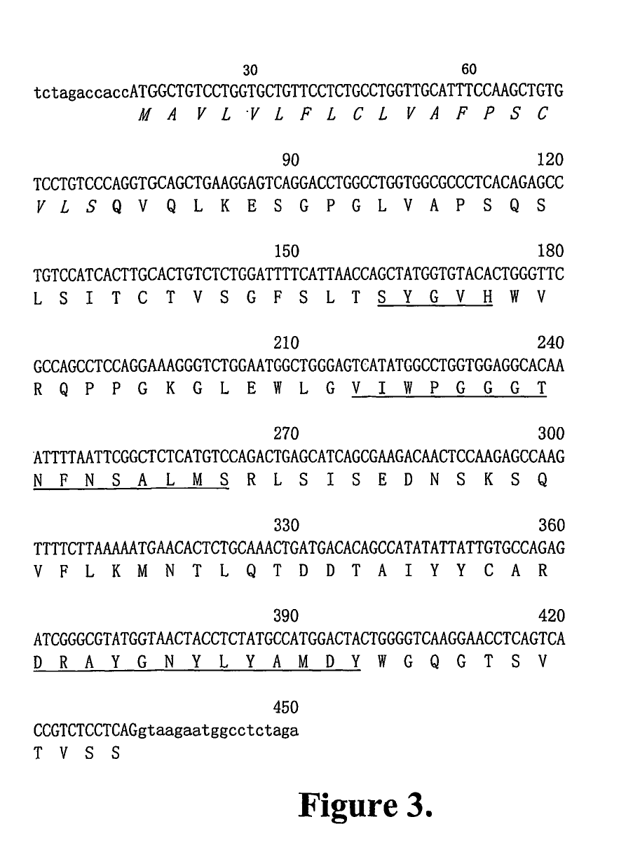

Several light and heavy chain clones were sequenced from two independent PCR reactions. For the light chain, two unique sequences homologous to mouse light chain variable regions were identified One VL sequence was non-functional due to a frame shift mutation and was identified as the non-productive allele. The other VL sequence was typical of a functional mouse kappa chain variable region. For the heavy chain, a unique sequence homologous to a typical mouse heavy chain variable region was identified. Their nucleotide sequences and their deduced amino acid sequences of variable region are described in FIG. 2 and FIG. 3.

### Example 3

### Construction and Expression of Chimeric TN228-IgG2M3

(Methods)

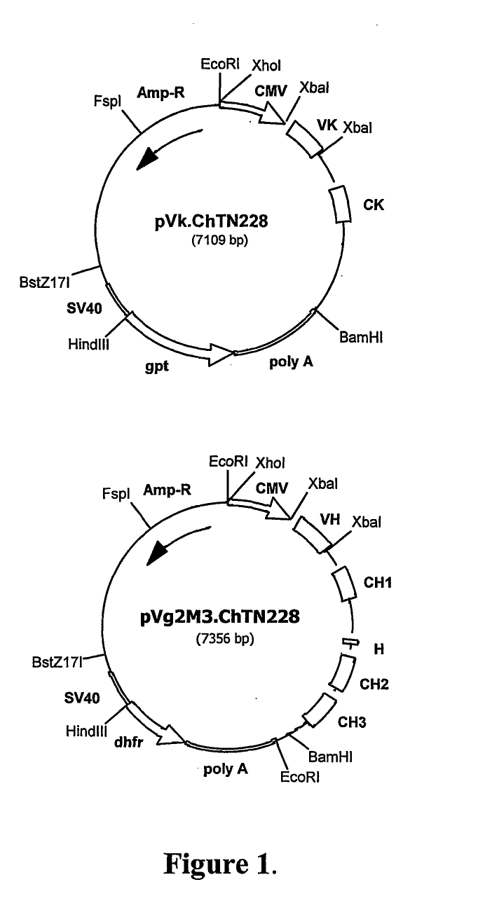

TN228 V_{L} and V_{H} were converted by PCR into mini-exon segments flanked by Xbal sites as described by He et al. (He, X. Y., Z. Xu, J. Melrose, A. Mullowney, M. Vasquez, C. Queen, V. Vexler, C. Klingbeil, M. S. Co, and E. L. Berg. 1998. Humanization and pharmacokinetics of a monoclonal antibody with specificity for both E- and P-selectin. J. Immunol. 160: 1029-1035) and were subcloned into the light chain and heavy chain expression plasmids (FIG. 1). Each mini-exon contains a signal peptide sequence, a mature variable region sequence and a splicing donor sequence derived from the most homologous mouse J chain gene. Such splicing donor sequences are used to splice the V region exon to the human antibody constant region. Each mini-exon was sequenced after it had been cloned into the expression vector to ensure the correct sequence was obtained and that no PCR errors were generated. The constant region exons of the light and heavy chain expression plasmids were also confirmed by sequencing.

In this specification, ChTN228 refers to a chimeric antibody containing the mouse TN228 VL and VH variable regions, a human IgG2M3 constant region for the heavy chain, and a human kappa constant region for the light chain. The heavy chain constant region was modified (Cole, M. S., C. Anasetti, and J. Y. Tso. 1997. Human IgG2 variants of chimeric anti-CD3 are nonmitogenic to T cells. J. Immunol. 159: 3613-3621) from the germline human 2 genomic fragment, and the light chain was derived from the germline human K genomic fragment. Both the heavy and light chain genes are driven by the human cytomegalovirus major immediate early promoter and enhancer. The heavy chain gene is followed by the transcription terminator derived from the human complement gene C2 (Ashfield, R., P. Enriquez-Harris, and N. J. Proudfoot. 1991. Transcriptional termination between the closely linked human complement genes C2 and factor B: common termination factor for C2 and c-myc? EMBO J. 10: 4197-4207). The light chain selection marker gpt gene (Mulligan, R. C., and P. Berg. 1981. Selection for animal cells that express the Escherichia coli gene coding for xantein-guanine phosphoribosyltransferase. Proc. Natl. Acad. Sci. USA 78: 2072-2076) and the heavy chain selection marker dhfr gene (Simonsen, C. C., and A. D. Levinson. 1983. Isolation and expression of an altered mouse dihydrofolate reductase cDNA. Proc. Natl. Acad. Sci. USA 80: 2495-2499) are both driven by the SV40 early promoter. For expression of chimeric TN228, transient transfection into COS-7 cells (monkey kidney cell line) was done using lipofectamine (catalog # 10964-013, GIBCO BRL). Spent media from transient transfectants were analyzed for human IgG2M3 antibody production by ELISA, using goat anti-human IgG gamma chain specific antibody as—capturing reagent and BRP-conjugated goat anti-human kappa chain antibody as developing reagent. The spent media was also tested for the ability of ChTN228 to bind to P815/CD28⁺ cells (stably transfected cell with CD28 into P815 (mouse mastcytoma)) by indirect immunofluorescent staining and analyzed by flow cytometry. For stable cell line-production, the chimeric expression plasmids were transfected into murine myeloma cell line Sp2/0 by electroporation and the transfectants were selected for gpt expression. The spent media from stable transfectants were analyzed by ELISA as for the transient transfection.

Results

The cloned V_{L} and V_{H} genes were converted into mini-exons by PCR (FIG. 2 and 3) and subcloned into the light and heavy chain expression vectors as described above and shown in FIG. 1.

Transient transfection of COS-7 cells: The chimeric expression vectors were transiently transfected into monkey kidney cell line COS-7 to produce the chimeric TN228⁺ antibody. Spent medium from the transfected cells was tested by ELISA for the production of chimeric IgG2M3 antibodies and by flow cytometry for binding to P815/CD28⁺ cells. Spent medium was positive in both assays. The yield of chimeric antibody from transient transfection was ˜0.9 μg/ml. The ChTN228 antibody from transient supernatant bound to P815/CD28⁺ cells in a concentration dependent manner (data not shown).

Stable transfection of Sp2/0 cells: The chimeric expression vectors were transfected into Sp2/0 cells for the production of a stable cell line. Spent media from several transfectants were tested for the production of chimeric TM228 antibody and for binding to P815/CD28⁺ cells as with the transient transfectants. Most transfectants were positive for both assays. One transfectant was chosen for its higher antibody productivity and expanded to grow in 5 L of serum free medium. ChTN228 was purified from 5 L of spent medium by affinity chromatography. The yield of purified antibody was ˜25 mg.

### Example 4

### Protein Purification of Chimeric Antibody ChTN228

One of the high ChTN228 expressing transfectants from the stable transfection (Clone 7H) was grown in 5 L of GIBCO hybridoma serum-free medium (catalog # 12045-076, GIBCO BRL). Spent culture supernatant was harvested when cell viability reached 10% or below, concentrated to 500 ml and loaded onto a 5 ml protein-A Sepharose column using a Pharmacia P1 pump (2-3 ml/min). The column was washed with PBS before the antibody was eluted with 0.1 M Glycine, 0.1 M NaCl, pH 2.7. The eluted protein was dialyzed against 3 changes of 2 L PBS and then desalted onto a PD-10 column equilibrated with PBS containing an additional 0.1 M NaCl. The desalted protein solution was filtered through a 0.2 μm filter prior to storage at 4 C.

### Example 5

### Purity Determination by Size Exclusion HPLC and SDS-PAGE

Size exclusion HPLC was performed using a Perkin Elmer BPLC system consisting of a PE ISS 200 Advanced LC Sample Processor, a PE Series 410 Bio LC Pump, a PE 235C Diode Array Detector, and a PE Nelson 600 Series LINK. Perkin Elmer Turbochrom Navigator Version 4.1 software was used to control the autosampler, pump, and detector, and to acquire, store, and process the data. Separation was achieved using two TosoHaas TSK-GEL G3000SWXL size exclusion HPLC columns, 7.8 mm×300 mm, 5 μm particle size, 250 Å pore size (catalog # 08541, TosoHaas, Montgomeryville, Md.) connected in series. The mobile phase was 200 mM potassium phosphate/150 mM potassium chloride at pH 6.9, and the flow rate was 1.00 mL/minute. The column eluate was monitored spectrophotometrically at both 220 mn and 280 nm The injection volume was 50 μL (50 μg) of the ChTN228 sample.

SDS-PAGE was performed according to standard procedures on a 4-20% gradient gel (catalog # EC6025, Novex, San Diego, Calif.).

The purity of the isolated ChTN228 was analyzed by size exclusion HPLC and SDS-PAGE. Based on this analysis, the protein is 96.5% monomer and has the mobility corresponding to a protein of molecular weight ˜160 kD. SDS-PAGE analysis of MuTN228, isotype control MuFd79 (mouse IgG1), ChTN228, and isotype control HuEP5C7 (human IgG2M3) under nonreducing conditions also indicated that all four antibodies have a molecular weight of about 150-160 kD. Analysis of the same four proteins under reducing conditions indicated all four antibodies were comprised of a heavy chain with a molecular weight of about 50 kD and a light chain with a molecular weight of about 25 kD.

### Example 6

### Competition Experiment

Methods

A titration experiment was done using serial two-fold dilutions of MuTN228-FITC antibody beginning at 250 ng/est. P815/CD28⁺ cells (510⁵ cells/test) were incubated with FITC-labeled antibody for 1 hour on ice washed with PBS and analyzed by flow cytometry. For the competition experiments, 25 ng of MuTN228-FITC and serial two-fold dilutions of competing ChTN228 or MuTN228 antibodies beginning at 800 ng/test were added to P815/CD28⁺ cells (5×10⁵ cells/test). As a control, P815/CD28⁺ cells (5×10⁵ cells/test) were incubated with 25 μng of MuTN228-FITC alone (i.e. without any competitor). HuEP5C7 and MuFd79 isotype control antibodies (800 ng/test) were also tested as competitors. Cells were incubated with the antibody mixture in a final volume of 150 μl for one hour on ice (in the dark), then washed and analyzed by flow cytometry.

Results

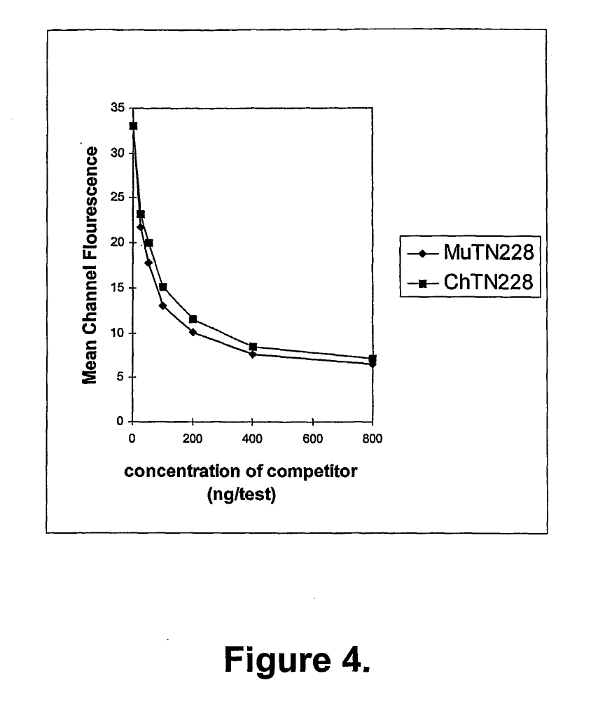

The binding specificity of the MuTN228 and ChTN228 antibodies was compared in a flow cytometry competition experiment as described in the Methods. Various amounts of unlabeled MuTN228 or ChTN228 were mixed with 25 ng of FITC-labeled MuTN228 antibody and incubated with P815/CD28′ cells. Both MuTN228 and ChTN228 competed with MuTN228-FITC in a concentration dependent manner, indicating that binding of both antibodies is specific for the CD28 antigen (FIG. 4). The isotype control antibodies MuFd79 and HuEP5C7 did not compete with MuTN228-FITC, indicating that the MuTN228 and ChTN228 antibodies recognize the CD28 antigen through V-region specific interactions.

### Example 7

### Chimeric Anti-human CD28 Antibody which has Reduced Affinity to Human F R Inhibits Primary Mixed Lymphocyte Reaction

Cell Preparation

Human peripheral blood mononuclear cells (PBMC) were prepared from normal healthy volunteers by density gradient centrifugation using Ficoll-Paque plus (Amersham Pharmacia Biotech, Tokyo, Japan). Human blood were diluted with equal volume of RPM11640 and overlaid on Ficoll-Paque plus. After centrifugation for 30 min. at room temperature, PBMCs were collected and washed with RPMI1640. Thereafter, PBMCs were suspended with the medium(RPMI1640 containing 2.5% human type AB serum, 2-mercaptoethanol, and antibiotics) and applied to a nylon fiber column(Wako junyaku, Osaka, Japan). After 1 hr incubation at 37 C in 5% CO₂, T cells were eluted with warm medium.

Human B cell lines (Raji and JY) were used as stimulator cells in the mixed lymphocyte reaction. These cells were X-ray irradiated (2000R) before use.

Primary Mixed Lymphocyte Reaction (1^{st} MLR)

Purified human T cells (1×10⁵ cells/well) and irradiated Raji (1×10⁵ cells/well) were plated in 96 well flat bottom micro plate. Antibodies were added to the culture medium and cells were incubated for 7 days. All cultures were labeled for final 6 hours with 10 kBq/well [³H]thymidine (Amersham Pharmacia biotech). Cells were harvested and incorporated radioactivity was measured by liquid scintillation counter.

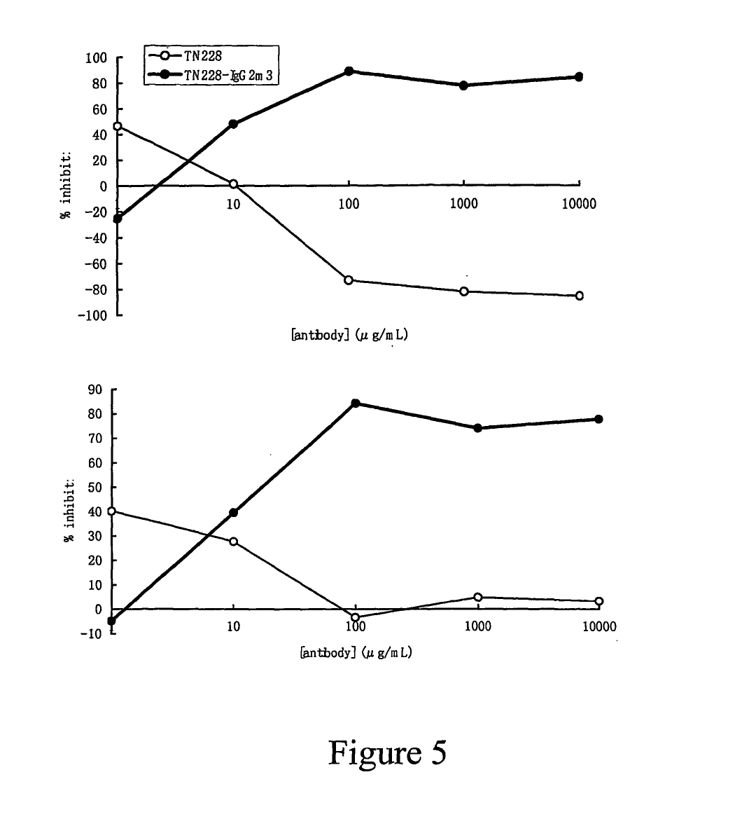

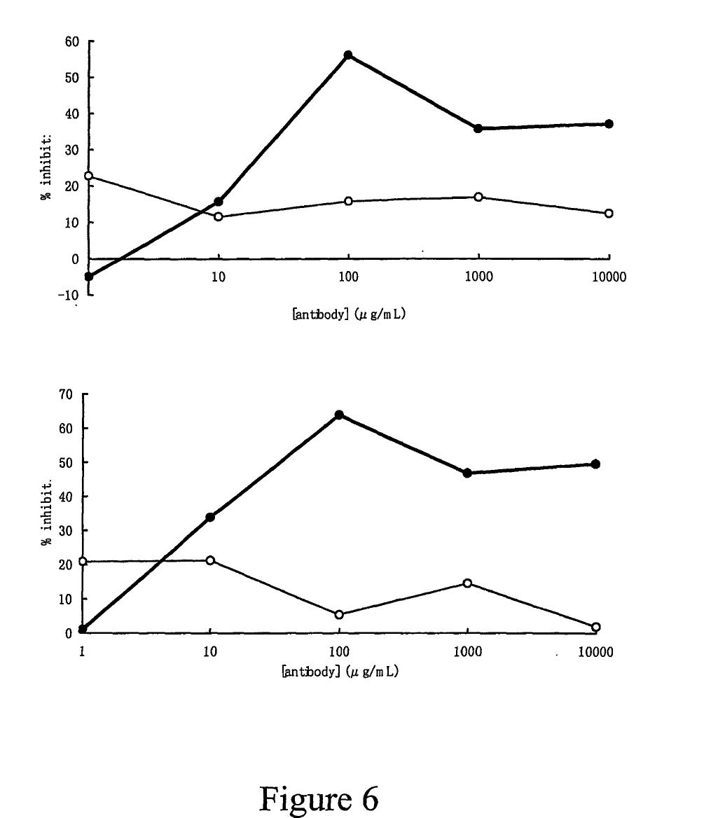

The effect of TN228-IgG2m3 (ChTN228) on primary MLR was shown on FIGS. 5 and 6. The original anti-human CD28 antibody TN228 (MuTN228) did not inhibit primary MLR, however, chimneric antibody TN228-IgG2m3 inhibited in a dose dependent manner. Therefore, conversion of Fc region of anti-human CD28 antibody to one with reduced affinity to human Fc R makes the antibody antagonistic to T cell proliferation.

Chimeric anti-human CD28 antibody which has reduced affinity to human Fc R reduced T cell low responsiveness in secondary mixed lymphocyte reaction.

Secondary Mixed Lymphocyte Reaction (2^{nd} MLR)

Purified human T cells (1×10⁵ cells/well) and irradiated Raji cells (1×10⁵ cells/well) were plated in 96-well flat bottom micro plates. Antibodies were added to the culture medium and cells were incubated. After 5 days, cells were collected, washed with fresh medium. Cells were, suspended with fresh medium and cultured for 8 days. Cells were restimulated with irradiated Raji or JY cells. After additional 7 days culture, cells were incubated with 10 kBq/well [³H]thymidine for 6 hours. Cells were harvested and radioactivity was measured by liquid scintillation counter.

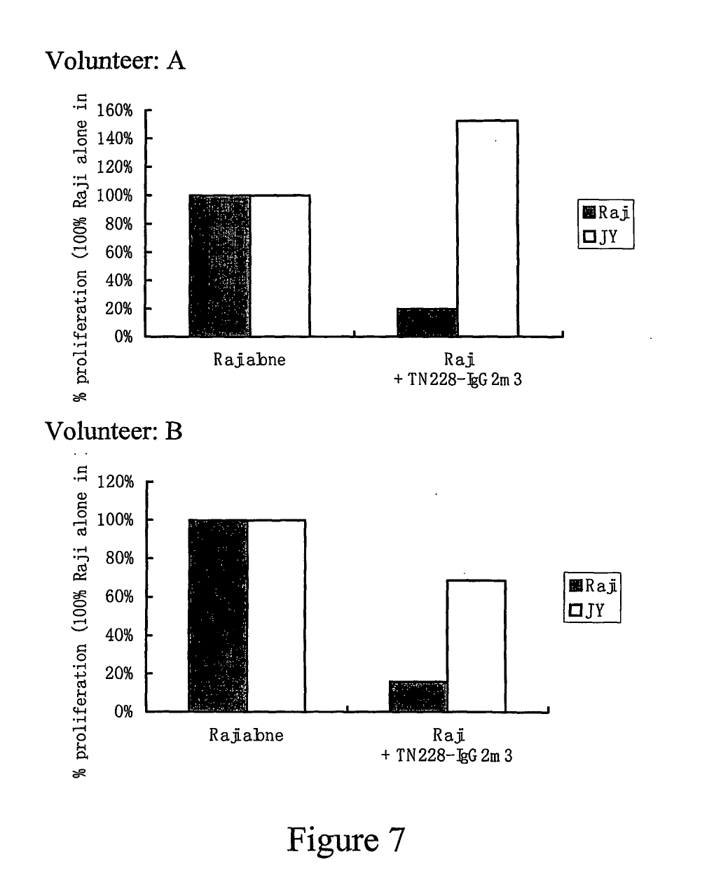

TN228-IgG2m3 inhibited primary MLR (FIGS. 5 and 6). Next, we analyzed the effect of this antibody on secondary MLR. The antibody was applied to primary MLR culture, then antibody was removed from culture supernatant After culturing in the medium without antibodies, cells were re-stimulated with the same stimulator cells(Raji) or third party stimulator(JY). The proliferation of cells treated with TN228-IgG2m3 through primary MLR was reduced compared to that of none-treated cells. However, both cells proliferated to almost the same extent with third party stimulator (JY) (FIG. 7). This result indicates that anti-human CD28 antibody with reduced affinity to human Fc R may induce T-ell energy through alo-antigen stimulations.

### Example 8

### Design of Humanized TN228 Variable Regions

The V-region sequences of MuTN228 were analyzed by computer modeling. Based on a sequence homology search against the Kabat antibody sequence database (8. Johnson, G., and T. T. Wu. 2000. Kabat database and its applications: 30 years after the first variability plot Nucleic Acids Res. 28: 214-218), IC4 (Manheimmer-Lory, A, J. B. Katz, M. Pillinger, C. Ghossein, A. Smith, B. Diamond. 1991. Molecular characteristics of antibodies bearing an anti-DNA-associated idiotype. J. Exp. Med. 174: 1639-1652) was selected to provide the framework for both the humanized TN228 heavy chain and light chain variable regions. The humanized TN228 heavy chain variable domain has 65 residues out of 85 framework residues that are identical to those of the mouse TN228 heavy chain framework, or 76% sequence identity. The humanized TN228 light chain variable domain has 56 residues out of 80 framework residues that are identical to those of the mouse TN228 light chain framework, or 70% sequence identity.

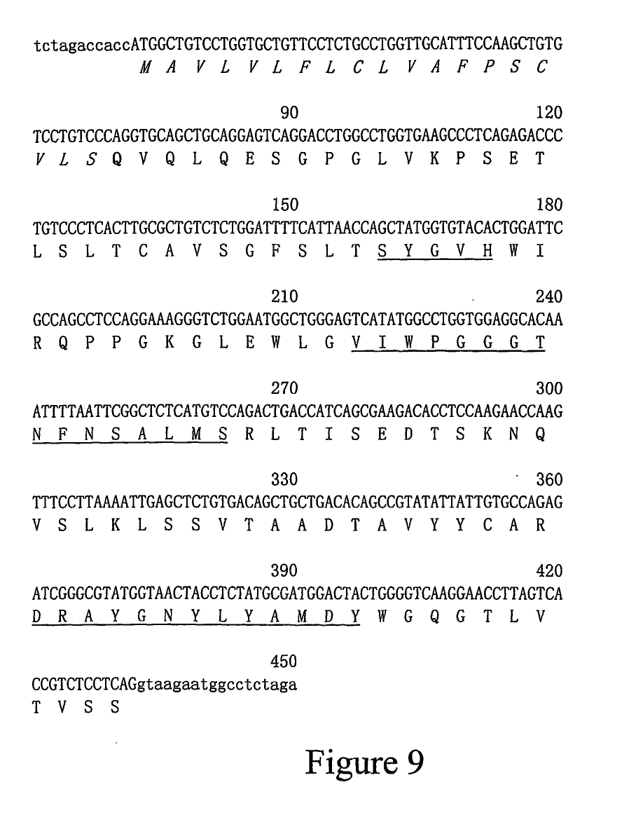

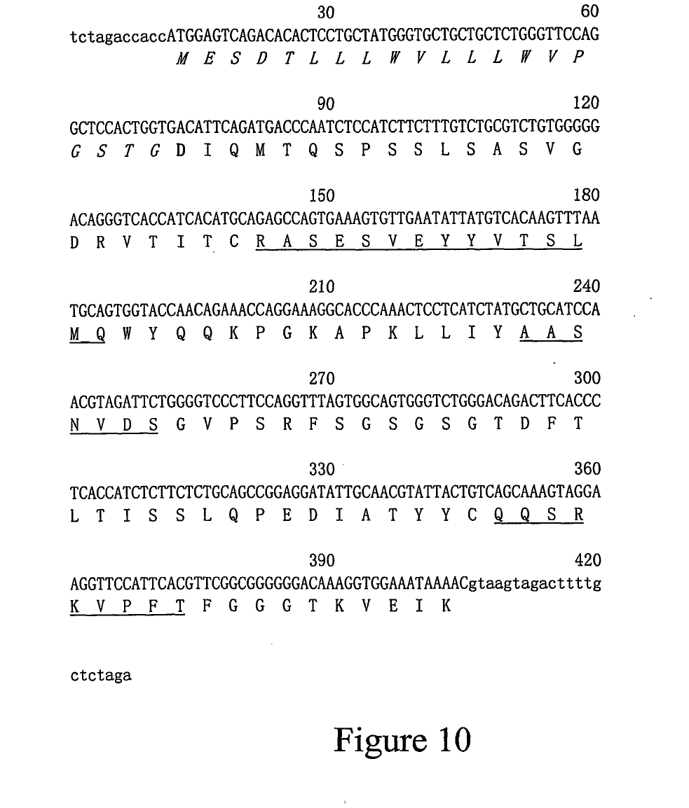

The computer programs ABMOD and ENCAD (Levitt, M. 1983. Molecular dynamics of native protein. I. Computer simulations of trajectories. J. Mol. Biol. 168: 595-620) were used to construct a molecular model of the TN228 variable domain, which was used to locate the amino acids in the mouse TN228 framework that are close enough to the CDRs to potentially interact with them. To design the humanized TN228 heavy and light chain variable regions, the CDRs from the mouse TN228 heavy chain were grafted into the framework regions of the human IC4 heavy chain and the CDRs from the mouse TN228 light chain were grafted into the framework regions of the human IC4 light chain. At framework positions where the computer model suggested significant contact with the CDRS, the amino acids from the mouse antibody were substituted for the original human framework amino acids. For humanized TN228, this was done at residues 27, 29, 30, 48, 67, 71 and 78 of the heavy chain. For the light chain, no substitutions were made (i.e., a straight grafting of the MuTN228 CDRs into the IC4 framework region was done). Furthermore, framework residues that occurred only rarely at their positions in the database of human antibodies were replaced by human consensus amino acids at those positions. For humanized TN228 this was done at residues 23, 40, 73, 83 and 85 of the heavy chain and at residues 69 and 77 of the light chain. The amino acid sequences of the humanized TN228 antibody heavy and light chain variable regions are shown FIG. 9 and 10.

### Example 9

### Construction and Expression of Humanized TN228-IgG2M3

Methods

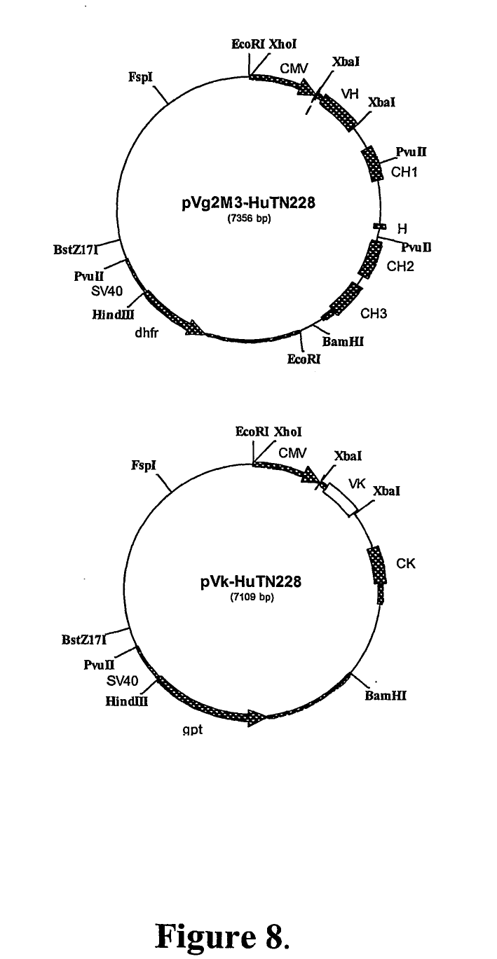

Once the humanized variable region amino acid sequences had been designed as described above, genes were constructed to encode them, including signal peptides, splice donor signals and appropriate restriction enzyme sites (FIG. 8). The heavy and light chain variable region genes were constructed and amplified using eight overlapping synthetic oligonucleotides ranging in length from approximately 65 to 80 bases (He, X. Y, Z. Xu, J. Melrose, A. Mullowney, M. Vasquez, C. Queen, V. Vexler, C. Klingbeil, M. S. Co, and E. L. Berg. 1998. Humanization and pharmacolinetics of a monoclonal antibody with specificity for both E- and P-selectin. J. Immunol. 160: 1029-1035). The oligonucleotides were annealed pairwise and extended with the Kienow fragment of DNA polymerase I, yielding four double-stranded fragments. The resulting fragments were denatured, annealed pairwise, and extended with Klenow, yielding two fragments. These fragments were denatured, annealed pairwise, and extended once again, yielding a full-length gene. The resulting product was amplified by polymerase chain reaction (PCR) using Taq polymerase, gel-purified, digested with Xbal, gel-purified again, and subcloned into the XbaI site of pVg2M3 for the expression of heavy chain, and pVk for the expression of light chain. The pVg2M3 vector for human gamma 2 heavy chain expression (Cole, M. S., C. Anasetti, and J. Y. Tso. 1997. Human IgG2 variants of chimeric anti-CD3 are nonmitogenic to T cells. J. Immunol. 159: 3613-3621), and the pVk vector for human kappa light chain expression (CO, M. S., N. M. Avadalovic, P. C. Caron, M. V. Avadalovic, D. A. Scheinberg, and C. Queen. 1992. Chimeric and humanized antibodies with specificity for the CD33 antigen. J. Immunol. 148:1149-1154) have been previously described.

The sequences of the V-regions and constant region exons of the heavy and light chain final plasmids were verified by nucleotide sequencing. The gross structures of the final plasmids were verified by restriction mapping. All DNA manipulations were performed by standard methods.

In this specification, HuTN228 refers to a humanized antibody containing the humanized TN228 V_{H} and V_{L} variable regions, a human IgG2M3 constant region for the heavy chain, and a human kappa constant region for the light chain. The heavy chain constant region was modified (Cole, M. S., C. Anasetti, and J. Y. Tso. 1997. Human IgG2 variants of chimeric anti-CD3 are nonmitogenic to T cells. J. Immunol. 159: 3613-3621) from the germline human 2 genomic fragment, and the light chain was derived from the germline human K genomic fragment. The human cytomegalovirus major immediate early promoter and enhancer drive both the heavy and light chain genes. The heavy chain gene is followed by the transcription terminator derived from the human complement gene C2 (Ashfield, R., P. Enriquez-Harris, and N. J. Proudfoot 1991. Transcriptional termination between the closely linked human complement genes C2 and factor B: common termination factor for C2 and c-myc? EMBO J. 10: 4197-4207). The light chain selection marker gpt gene (Mulligan, R. C., and P. Berg. 1981. Selection for animal cells that express the Escherichia coli gene coding for xanthine-guanine phosphoribosyltransferase. Proc. Natl. Acad. Sci. USA 78: 2072-2076) and the heavy chain selection marker dhfr gene (Simonsen, C. C., and A. D. Levinson. 1983. Isolation and expression of an altered mouse dihydrofolate reductase cDNk Proc. Natl. Acad. Sci. USA 80: 2495-2499) are both driven by the SV40 early promoter.

For expression of HuTN228, transient transfection into COS-7 cells (monkey kidney cell line) was done using Lipofectamine 2000 (catalog # 11668-027, Life Technologies). Spent media from transient transfectants were analyzed for human IgG2M3 antibody production by ELISA, using goat anti-human IgG gamma chain specific antibody as capturing reagent and HRP-conjugated goat anti-human kappa chain antibody as developing reagent. The spent media were also tested for the ability of HuTN228 to bind to P815/CD28⁺ cells by indirect immunofluorescent staining and analyzed by flow cytometry (data not shown). For stable cell line production, the humanized expression plasmids were transfected into murine myeloma cell line Sp2/0 by electroporation and the transfectants were selected for gpt expression. The spent media from stable transfectants were analyzed by ELISA as for the transient transfection.

Results

Based on the humanized V-region amino acid sequence design, heavy and light chain V-genes (FIG. 9 and 10) were constructed as described in the Methods. The heavy and light chain V-genes were cloned into the pVg2M3 and pVk vectors, respectively, as shown in FIG. 8. Several clones were analyzed by nucleotide sequencing and correct clones of both-the heavy chain and light chain expression vectors were used for the transfection. The constant regions of both the heavy and light chain expression vectors were also confirmed by sequencing.

### Example 10

### Expression of HuTN228

Transient transfection of COS-7 cells: The expression vectors were transiently transfected into monkey kidney cell line COS-7 to produce the HuTN228 antibody. Spent medium from the transfected cells was tested by ELISA for the production of humanized gG2M3 antibodies and by flow cytometry for binding to ^{P815}/CD28⁺ cells (data not shown). Spent medium was positive in both assays. The yield of humanized antibody from transient transfection was −3.7 g/ml. The HuTN228 antibody from transient supernatant bound to P815/CD28⁺ cells in a concentration dependent manner (data not shown).

Stable transfection of Sp2/0 cells: The humanized expression vectors were transfected into Sp2/0 cells for the production of a stable cell line. Spent media from several transfectants were tested for the production of HuTN228 antibody as with the transient transfectants. One transfectant (clone 4) was chosen for its higher antibody productivity and expanded in GIBCO hybridoma serum free medium. HuTN228 antibody was purified from 570 ml of spent medium by affinity chromatography. The yield of purified antibody was ˜7 mg.

### Example 11

### Protein Purification

One of the high HuTN228 expressing transfectants from the stable transfection (Clone 4) was grown in 570 ml of GIBCO hybridoma serum free medium (catalog # 12045076, Life Technologies). Spent culture supernatant was harvested when cell viability reached 10% or below and loaded onto a 2 ml protein-A Sepharose column. The column was washed with PBS before the antibody was eluted with 0.1 M Glycine, 0.1 M NaCl, pH 2.5. The eluted protein was dialyzed against 3 changes of 2 L PBS and then desalted onto a PD-10 column equilibrated with PBS containing an additional 0.1 M NaCl. The desalted protein solution was filtered through a 0.2 m filter prior to storage at 40C.

### Example 11

### Purity Determination by Size Exclusion HPLC and SDS-PAGE

Methods

Size exclusion HPLC was performed using a Perkin Elmer HPLC system consisting of a PE ISS 200 Advanced LC Sample Processor, a PE Series 410 Bio LC Pump, a PE 235C Diode Array Detector, and a PE Nelson 600 Series LINK. Perkin Elmer Turbochrom Navigator Version 4.1 software was used to control the autosampler, pump, and detector, and to acquire, store, and process the data. Separation was achieved using two TosoHaas TSK-GEL G3000SWXL size exclusion HPLC columns (7.8 mm×300 mm, 5 m particle size, 250 pore size; catalog #08541, TosoHaas, Montgomeryville, Md.) connected in series. The mobile phase was 200 mM potassium phosphate/150 mM potassium chloride at pH 6.9, and the flow rate was 1.00 mL/minute. The column eluate was monitored spectrophotometrically at both 220 nm and 280 mn. The injection volume was 60 1 (60 g) of the HuTN228 sample.

SDS-PAGE was performed according to standard procedures on a 4-20% gradient gel (catalog # EC6025, Novex, San Diego, Calif.).

The isotype of the purified antibody was confirmed using the Human IgG Subclass Profile ELISA Kit (catalog #99-1000, Zymed Laboratories, South San Francisco, Calif.) following the manufacturer's recommendations. (Results)

The purity of the isolated HuTN228 antibody was analyzed by size exclusion HPLC and SDS-PAGE. The HPLC elution profile of HuTN223 is not shown. Based on this analysis, the protein is −98% monomer and has the mobility corresponding to a protein of molecular weight −160 kD.

SDS-PAGE analysis of MuTN228, isotype control MuFd79 (mouse IgG1), HuTN228, and isotype control HuEP5C7 (human IgG2M3) under nonreducing conditions also indicated that all four antibodies have a molecular weight of about 150-160 kD. Analysis of the same four proteins under reducing conditions indicated that all four antibodies were comprised of a heavy chain with a molecular weight of about 50 kD and a light chain with a molecular weight of about 25 kD.

The isotype test indicated that the isotype of the HuTN228 antibody was consistent with the expected IgG2 isotype (data not shown).

### Example 12

### FACS Competition Experiment

Methods

A titration experiment was done using serial two-fold dilutions of MuTN228-FITC antibody beginning at 250 ng/test P815/CD28⁺ cells (3×1O⁵ cells/test) were incubated with FITC-labeled antibody for 1 hour on ice in 100 1 of FACS Staining Buffer (FSB=PBS, 2% FBS, 3% normal mouse serum, 0.1% NaN₃) washed with 2 ml of FSB, and analyzed by flow cytometry (data not shown).

For the competition experiments, MuTN228-FITC (50 ng/test) in 25 1 of FSB was combined with three-fold serial dilutions of competing HuTN228 or MuTN228 antibodies (beginning at 200 g/ml) in 25 1 of FSB, and added to P815/CD28⁺ cells (3×10⁵ cells/test) in 50 1 of FSB. As a control, P815/CD28⁺ cells were incubated with MuTN228-FITC alone (50 ng/test in 50 1 of FSB). HuEP5C7 (human IgG2M3) and MuFd79 (mouse IgG1) isotype control antibodies (200 g/ml) in 25 1 of FSB were also tested as nonspecific competitors. Cells were incubated with the antibody mixture in a final volume of 100 1for one hour on ice (in the dark), then washed with 2 ml of FSB, and analyzed by flow cytometry. This experiment was repeated three times.

Results

The binding specificity of the MuTN228 and HuTN228 antibodies to CD28 molecules on P815/CD28⁺ cells was compared in a flow cytometry competition experiment as described in the Methods. A representative result is shown in FIG. 5. Both MuTN228 and HuTN228 competed with MuTN228-FITC in a concentration dependent manner, indicating that binding of both antibodies is specific for the CD28 antigen. The relative binding of HuTN228 was a few fold less than that of MuTN228. The isotype control antibodies MuFd79 and HuEP5C7 did not compete with MuTN228-FITC, indicating that the MuTN228 and HuTN228 antibodies recognize the CD28 antigen through V-region specific interactions.

### Example 13

### ELISA Competition Experiment

Methods

A 96 well ELISA plate (Nunc-Immuno plate, catalog # 439454, NalgeNunc, Naperville, Ill.) was coated with 100 1/well of sCD28-Fc (0.5 g/ml in PBS) (sCD28-Fc means the fused protein, in which the extracellular domains of CD28 were combined with the CH2 and CH3 domains of IgG1.) overnight at 4 C. The plate was blocked for 30 minutes with 300 1/well of Superblock Blocking Buffer in TBS (catalog # 37535, Pierce, Rockford, Ill.), and washed with 300 1/well of ELISA WashBuffer (EWB=PBS, 0.1% Tween-20). A mixture of MuTN228-biotin (0.5 g/ml) in 100 1 of ELISA Buffer (EB=PBS, 1% BSA, 0.1% Tween-20) and three-fold serially diluted HuTN228 or MuTN228 competitor antibodies (starting at 100 g/ml) in 100 1 of EB was added in triplicate in a final volume of 200 1/well. Isotype control antibodies HuEP5C7 and MuFd79 (100 g/ml) in 100 1 of EB were also tested as non-specific competitors. As a ‘no competitor’ control, 100 1 of EB was added to 100 1of MuTN228-biotin (0.5 g/ml). As a blank, 200 1 of EB was added to the remaining wells (containing no MuTN228-biotin). The plate was incubated at room temperature for 1.5 hours with shaking. After washing the wells 4 times with 300 1/well of EWB, 100 1/well of Streptavidin-HRP (1 g/ml, catalog # 21124, Pierce) was added to all the wells. The plate was incubated at room temperature for 1 hour with shaking. After washing the wells as above, 100 1/well of ABTS substrate (catalog #507602 & 506502, KPL, Gaithersburg, Md.) was added to all the wells. The plate was incubated at room temperature for 5-7 minutes and the optical density was read at 415 nm. This experiment was repeated three times.

Results

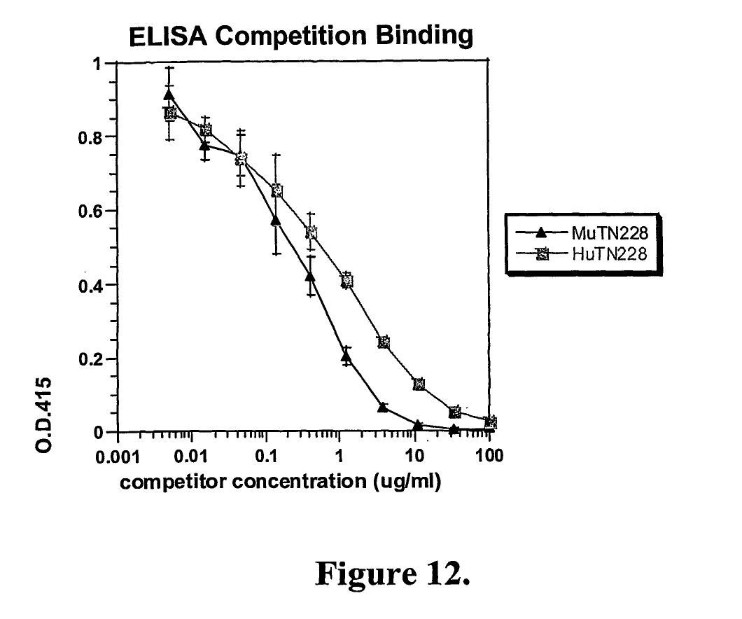

The binding specificity of the HuTN228 and MuTN228 antibodies to sCD28-Fc was compared in an ELISA competition experiment as described in the Methods. A representative result is shown in FIG. 12. Both MuTN228 and HuTN228 competed with MuTN228-biotin in a concentration dependent manner. The isotype control antibodies MuFd79 and HuEP5C7 did not compete with MuTN228-biotin, indicating that the MuTN228 and HuTN228 antibodies recognize the CD28 antigen through V-region specific interactions. The IC₅₀ values of MuTN228 and HuTN228 for all three experiments are shown in Table 2. The relative binding of HuTN228 was on average 2.6 fold less than that of MuTN228.

TABLE 2
ELISA competition summary

| | IC₅₀ (g/ml) | | | | |
|---|---|---|---|---|---|
| Competitor | Expt 1 | Expt 2 | Expt 3 | Average | Std. Dev. |
| MuTN228 | 0.21 | 0.20 | 0.15 | 0.19 | 0.03 |
| HuTN228 | 0.37 | 0.64 | 0.48 | 0.50 | 0.14 |

### Example 14

### ¹²⁵I-labeled Antibody Competition Experiment

Methods

The relative binding affinities of the MuTN228 and HuTN228 antibodies were determined following the method of Queen et al. (Queen, C., W. P. Schneider, H. E. Selick, P. W. Payne, N. P. Landolfi, J. F. Duncan, N. M. Avdalovic, M. Levitt, R. P. Junghans, T. A. Waldmann. 1989. A humanized antibody that binds to the interleukin 2 receptor. Proc. Natl. Acad. Sci. 86:10029-10033). Briefly, ˜10 ng of ¹²⁵I-labeled MuTN228 in 50 1 of Binding Buffer (BB=PBS, 2% FBS, 1 g/ml mouse IgG, 0.1% NaN₃) was combined in triplicate with three-fold serial dilutions of MuTN228 or HuTN228 competitor antibodies (beginning at 400 g/ml) in 50 1 of BB, added to 100 1 of P815/CD28⁺ cells (2.5×10⁵ cells/test) in incubation tubes (Skatron Macrowell Tube Strips, catalog # 15773, Molecular Devices, Sunnyvale, Calif.), and incubated for 90 minutes at 4 C with gentle shaking. Isotype control antibodies HuEP5C7 and MuFd79 (400 g/ml) in 50 1 of BB were also tested as nonspecific competitors. Following the incubation, the cell-antibody mixture was transferred to centrifuge tubes (Sarstedt Micro Tubes, catalog # 72.702, Sarstedt, Newton, N.C.) containing 0.1 ml 80% dibutyl phthalate-20% olive oil, the incubation tubes were washed once with 50 1 of BB, and bound and free counts were separated by centrifugation as described (Kuziel, W. A., S. J. Morgan, T. C. Dawson, S. Griffin, O. Smithies, K. Ley, N. Maeda. 1997. Severe reduction in leukocyte adhesion and monocyte extravasation in mice deficient in CC chemokine receptor 2. Proc. Natl. Acad. Sci. 94:12053-12058). This experiment was repeated three times.

Results

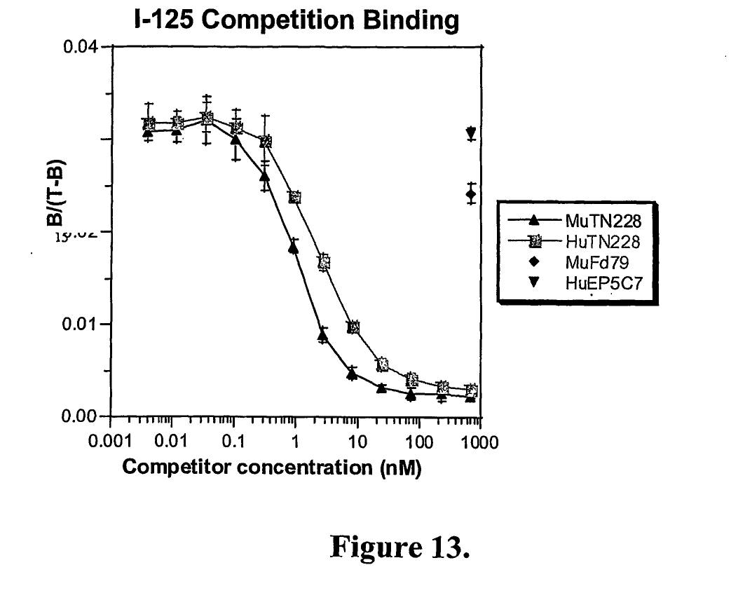

The relative binding affinities of the MuTN228 and HuTN228 antibodies were compared in an ¹²⁵I-labeled antibody competition experiment as described in the Method. A representative result is shown in FIG. 13. Both MuTN228 and HuTN228 competed with ¹²⁵I-labeled MuTN228 in a concentration dependent manner. The isotype control antibody MuFd79 showed weak but repeatable competition at high concentrations, but the isotype control antibody HuEP5C7 did not compete with ¹²⁵I-labeled MuTN228, indicating that the HuTN228 antibody recognizes the CD28 antigen through V-region specific interactions. The IC₅₀ values of MuTN228 and HuTN228 for all three experiments are shown in Table 3. The apparent binding affinity of HuTN228 was approximately 2.4 fold less than that of the MuTN228 antibody.

TABLE 3
I-125 competition summary

| | IC₅₀ (nM) | | | | |
|---|---|---|---|---|---|
| Competitor | Expt 1 | Expt 2 | Expt 3 | Average | St. Dev. |
| MuTN228 | 0.93 | 1.05 | 1.02 | 1.00 | 0.06 |
| HuTN228 | 2.65 | 2.43 | 2.13 | 2.40 | 0.26 |

### Example 15

### Amino Acid Sequencing of the Hamster Anti-murine CD28 Antibody

Method

Hybridoma and antibodies. The Armenian hamster anti-murine CD28 hybridoma PV1 was obtained from ATCC (ATCC HB-12352). Purified PV1, R-phycoerythrin (R-PE)-conjugated PV1 were purchased from Southern Biotechnology (Birmingham, Ala.). The Syrian hamster anti-CD28 antibody 37.51 was from PharMingen (San Diego, Calif.). Secondary antibodies fluorescein (FITC)-conjugated donkey anti-Armenian hamster IgG (H+L), FITC-conjugated donkey anti-Syrian hamster IgG (H+L), FITC-conjugated donkey anti-mouse IgG (H+L), R-PE-F(ab′)₂ donkey anti-mouse IgG (H+L) were from Jackson ImmunoResearch (West Grove, Pa.); and FITC-conjugated goat anti-mouse kappa, R-PE-conjugated goat anti-mouse IgG3, and horse radish peroxidase (HRP)-conjugated goat anti-mouse kappa were from Southern Biotechnology. Goat anti-mouse IgG3, and mouse IgG3 isotype control FLOPC 22 were from Sigma Chemicals (St. Louis, Mo.). The Armenian hamster anti-murine CD3 antibody 145.2C11 and its hamster/mouse chimeric version 145.2C11-IgG3 were generated in our laboratory. FITC-conjugated 145.2C11 was from Boehringer Mannheim (Indianapolis, Ind.).

Cloning of variable region cDNAs. The V region cDNAs for the light and heavy chains of PV1 were cloned from the hybridoma cells by an anchored polymerase chain reaction (PCR) method described by Co et al. (Co, M. S., N. M. Avadalovic, P. C. Caron, M. V. Avadalovic, D. A. Scheinberg, and C. Queen. 1992. J. Immunol. 148:1149-1154.). Amplification was performed on cDNA using 3′ primers that anneal respectively to the hamster kappa and gamma chain C regions, and a 5′ primer that anneals to the added G-tail of the cDNA. For V_{L}PCR, the 3′ primer has the sequence of 5′TATAGAGCTCCACTTCCAGTGCCC (SEQ ID NO:20), with residues 11-24 hybridizing to the hamster Cₖ region For V_{H} PCR, the 3′ primers has the degenerated sequences of (SEQ ID NOS:17, 18 and 19):

```
                                                            A G T
5′ TATAGAGCTCAAGCTTCCAGTGGATAGACCGATGGGGCTGTCGTTTTGGC,
                                                            T
```

with residues 19-50 hybridizing to most rodent IgG C_{H}1. The non-hybridizing sequences in the two primer sets contain restriction sites used for cloning. The V_{L} and V_{H} cDNAs were subcloned into a pUC19 vector for sequence determination. To avoid PCR-generated errors, five independent clones for each cDNA were sequenced, and only the clones whose sequence agreed with the consensus sequence were chosen to express the chimeric PV1.

Results

Cloning of PV1 V region cDNAs. The PV1 light and heavy chain V region cDNAs were cloned from the hybridoma cells as described in Methods. For the V_{L} PCR, only 3′ primer corresponding to the hamster C_{γ} region could yield V_{L} cDNA product from PV1. A 3′ primer from the hamster C_{γ} region, on the other hand, did not yield any PCR product. These results indicated that the hybridoma PV1 uses kappa for its light chain. Several light and heavy chain clones were sequenced and were found to contain the same V_{L} and V_{H}, respectively. Limited C_{H}1 and C_{γ} sequence data indicated that the cloned heavy and light chains are not murine in origin.

### Example 16

### Construction and Expression of Chimeric PV1-IgG3

Method

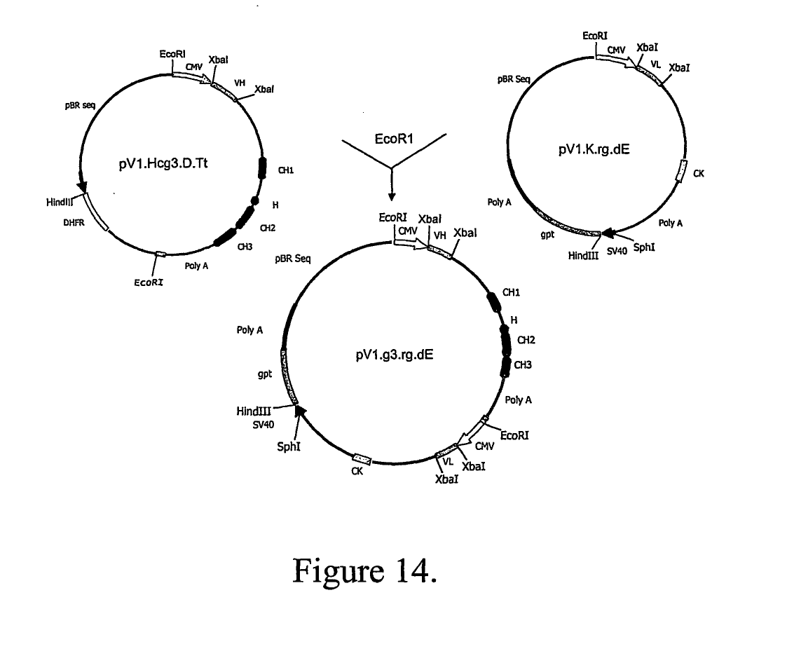

PV1 V_{L} and V_{H} were made by PCR into mini-exon segments flanked by XbaI sites as described (He, X. Y., Z. Xu, J. Melrose, A. Mullowney, M. Vasquez, C. Queen, V. Vexler, C. Klingbeil, M. S. Co, and E. L. Berg. 1998. J. Immunol. 160:1029-1035.) and they were separately introduced to the light chain and heavy chain expression plasmids (FIG. 14). Each mini-exon contains a signal peptide sequence, a mature variable region sequence and a 5′ splicing donor sequence derived from the most homologous mouse J chain gene. Such splicing donor is used to splice the V region exon to the mouse antibody constant region. Each mini-exon was sequenced again after it had been cloned into the expression vector to ensure the correct splicing signal was introduced, and no PCR errors were generated.

A vector was constructed to express both the heavy and light chain genes of the chimeric PV1-IgG3 from a single plasmid. In this report, PV1-IgG3 refers to a chimeric antibody containing the hamster PV1 V_{L} and V_{H} variable regions, a mouse IgG3 constant region for the heavy chain, and a mouse kappa constant region for the light chain. The expression vector pV1.g3.rg.dE (FIG. 14) was obtained by a two-step cloning process similar to that described by Cole et. al. (Cole, M. S., C. Anasetti, and J. Y. Tso. 1997. J. Immunol. 159:3613-3621.). The heavy chain constant region was derived from the mouse γ3 genomic fragment, and die light chain from the κ fragment. Both the heavy and light chain genes are driven by the human cytomegalovirus major immediate early promoter and enhancer, and they are separated by the transcription terminator derived from the human complement gene C2 (Ashfield, R., P. Enriquez-Harris, and N. J. Proudfoot. 1991. EMBO J. 10:4197-4207.). The selection marker gpt gene (Mulligan, R. C., and P. Berg. 1981. Proc. Natl. Acad. Sci. USA 78:2072-2076) is driven by a modified SV40 early promoter. For expression of the chimeric PV1-IgG3, the single plasmid vector was transfected into the murine myeloma cell line NS0, and the transfectants were selected for gpt expression. Spent media from transfectants were analyzed for mouse IgG3 antibody production by ELISA, using goat anti-mouse IgG3 as capturing reagent and HRP-conjugated goat anti-mouse kappa chain as developing reagent. The assay is specific for mouse IgG3; other mouse IgG isotypes are negative in this analysis.

Results

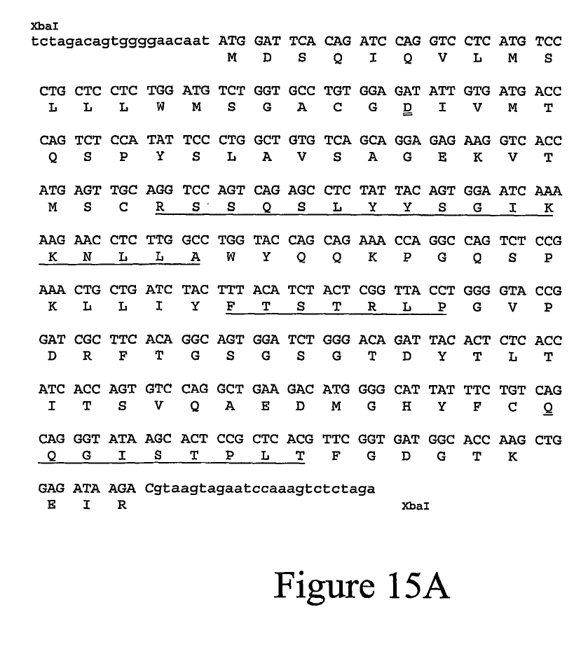

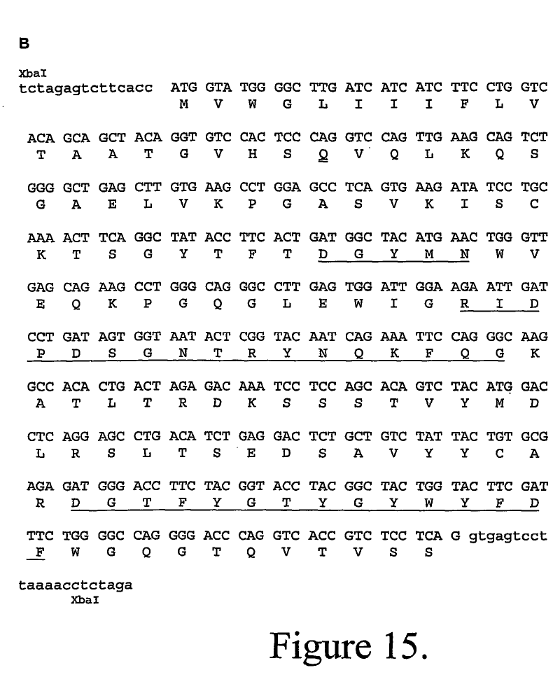

Expression of the chimeric PV1-IgG3. The cloned V_{L} and V_{H} were made into mini-exons (FIG. 15) and incorporated into an expression vector as described in Materials and Methods and FIG. 15. The expression vector was then transfected into a murine myeloma cell line NS0 to produce the chimeric PV1-IgG3. Spent media from several transfectants were assayed by ELISA for the production of mouse IgG3 antibodies and by FACScan for binding to EL4 cells. Most transfectants were positive in both assays. One transfectant was chosen for its high antibody productivity and expanded to grow in 1 L of serum-free medium. PV1-IgG3 was purified from the 1 L spent medium by affinity chromatography. The yield was >10 mg/L.

### Example 17

### Characterization of the Purified Chimeric PV1-IgG3 by HPLC and SDS-PAGE

Methods

Protein Purification. One of the high IgG3-expressing transfectants (Clone #1) was grown in 1 L of Gibco Serum-free Hybridoma medium. Spent culture supernatant was harvested when cell viability reached 30% or below, concentrated to 200 ml, and loaded onto a 5 ml protein-A Sepharose column using a Pharmacia P1 pump (2-3 ml/min). The column was then washed with PBS containing an additional 0.1 M NaCl (final concentration of NaCl was 0.25 M) before the antibody was eluted with 3.5 M MgCl₂. The eluted protein was then desalted onto a PD10 column equilibrated with PBS containing an additional 0.1 M NaCl. The desalted protein solution was filtered through a 0.2 μm filter prior to storage at 4° C. Like all mouse IgG3, PV1-IgG3 at high concentrations (>1 mg/mL) precipitates in the cold but returns to solution by warming at 37° C. The antibody stays in solution at room temperature. Repeated cycles of cold precipitation do not seem to affect the antigen binding activity of the antibody.

Purity Determination by size exclusion HPLC and SDS-PAGE. Size exclusion HPLC was performed using a Perkin Elmer HPLC system consisting of a PE ISS 200 Advanced LC Sample Processor, a PE Series 410 Bio LC Pump, a PE 235C Diode Array Detector, and a PE Nelson 600 Series LINK. Perkin Elmer Turbochrom Navigator Version 4.1 software was used to control the autosampler, pump, and detector, and to acquire, store, and process the data. Separation was achieved using two TosoHaas TSK-GEL G3000SWXL size exclusion HPLC columns (TosoHaas, catalog # 08541, 7.8 mm×300 mm, 5 μm particle size, 250 Å pore size) connected in series. The mobile phase was 200 mM potassium phosphate/150 mM potassium chloride at pH 6.9, and the flow rate was 1.00 mL/minute. The column eluate was monitored spectrophotometrically at both 220 nm and 280 nm The injection volume was 50 μL (63.5 μg) of the undiluted PV1-IgG3 sample. SDS-PAGE was performed according to standard procedures.

Results

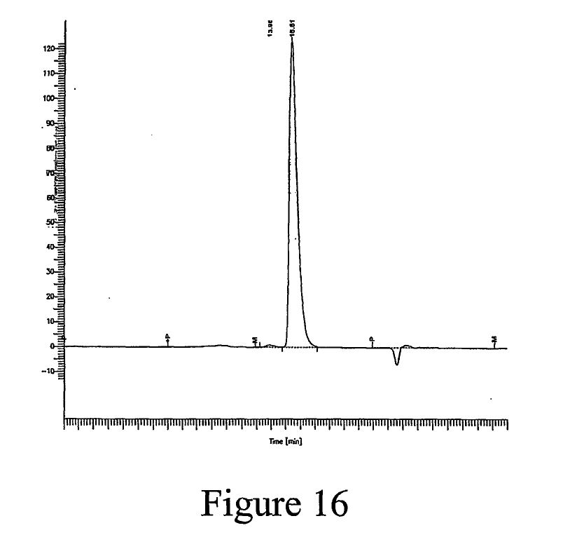

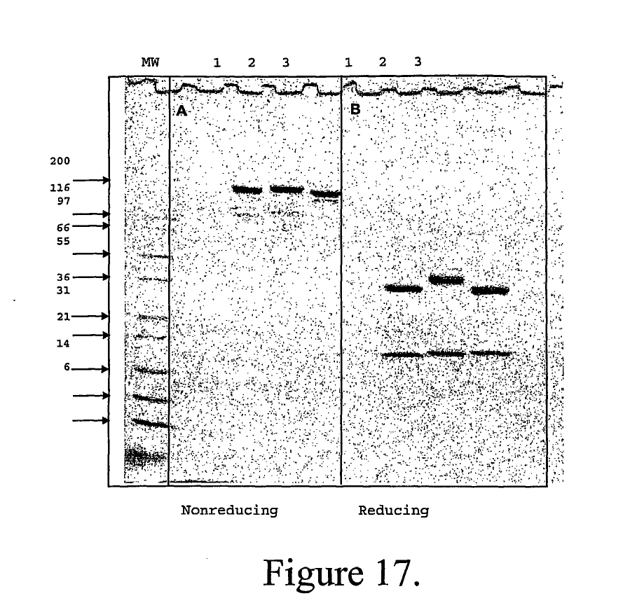

The purity of the isolated PV1-IgG3 was analyzed by size exclusion HPLC and SDS-PAGE. The HPLC elution profile of PV1-IgG3 is shown in FIG. 16. Based on this analysis, the protein is 99% monomer and has the mobility corresponding to the molecular weight of 150 kD. SDS-PAGE analysis of PV1, PV1-IgG3 and isotype control under nonreducing conditions also indicated all three antibodies have the molecular weight of about 150 kD (FIG. 17A). The minor bands seen in FIG. 17A were artifacts due to boiling of the samples in SDS without reduction. They reflected the number of incomplete inter-chain disulfide bonds in the antibodies. Analysis of the same three proteins under reducing conditions (FIG. 17B), however, indicated that PV1, but not PV1-IgG3 or the isotype control, has a heavy chain with molecular weight slightly higher tan the 50 kD molecular weight usually seen with IgG. The hamster antibody PV1 thus either has heavy glycosylation at Asn₂₉₇ in C_{H}3, or it has an extra glycosylation site elsewhere in the heavy chain. As discussed later, this unusual glycosylation pattern may contribute to PV1's nonspecific binding to EL4 cells, perhaps by lectin/carbohydrate interaction.

### Example 18

Methods

Flow cytometry. Murine T cell line EL4 cells (2.5×10⁵ cells/0.2 ml) were stained with 1 μg/ml of PV1, 37.51 or PVI-IgG3 at 4° C. for 30 min, washed with 2 ml of cold PBS, and stained with 20 μl of specific fluorochrome-conjugated secondary antibodies (10 μg/ml). After 20 min of incubation at 4° C. in the dark, the cells were washed with PBS and analyzed by FACScan (Becton Dickenson, Milpitas, Calif.).

In the competition experiment, EL4 cells (2.5×10⁵ cells/0.2 ml) were stained with 1 μg/ml of R-PE-PV1 and 25 μg/ml of PV1, PV1-IgG3, or IgG3 isotype control at 4° C. for 30 min in the dark, washed with PBS a analyzed by FACScan. Similar competition experiment was also conducted using various versions of 145.2C11. In the reverse competition experiment, EL4 cells (2.5×10⁵ cells/0.2 ml) were stained with 1 μg/ml of PV1-IgG3 and 25 μg/ml of PV1 at 4° C. for 30 min. washed 2 times with PBS, stained with FITC-conjugated donkey anti-mouse IgG (H+L), washed, and analyzed by FACScan. To control for nonspecific binding of the secondary antibodies to PV1, EL4 cells were stained with excess PV1 without PV1-IgG3 and analyzed.

For mouse T cell staining, BALB/c mouse splenic cells (2.5×10⁵ cells/0.2 ml) were stained with 1 μg/ml of mouse IgG3 isotype control (FLOPC 21) or PV1-IgG3 at 4° C. for 30 min, washed with 2 ml of cold PBS, and stained with 20 μl of FITC-conjugated 145.2C11 (10 μg/l) and 20 μl of R-PE-conjugated goat anti-mouse IgG3 (10 μg/ml). After 20 min of incubation at 4° C. in the dark, the cells were washed with PBS and analyzed by FACScan.

Results

Characterization of PV1 and PV1-IgG3 by flow cytometry. PV1 was used to stain CD28-positive T cell line EL4 and analyzed by FACScan. The pattern of staining indicated that PV1 binds EL4 cells at two different sites (FIG. 17A). In addition, PV1 as well as several Armenian hamster anti-murine T cell antibodies (145.2C11, anti-CD3; H57-597, anti-TCR; and UC10-4F10-11, anti-CTLA4) also bind nonspecifically to CD28-negative myeloma cell line NS0 (data not shown). The Syrian hamster anti-CD28 antibody 37.51, on the other hand, binds specifically to only one site on EL4 cells (FIG. 17B). It appears that, in addition to CD28 binding, PV1 also binds nonspecifically to other sites, possibly through the carbohydrate/lectin type of interaction. As shown in FIG. 17C, the chimeric PV1-IgG3 does not contain this nonspecific binding activity. The antibody binds EL4 cells in a pattern similar to that of 37.51, and it does not bind to CD28-negative NSO cells (data not shown). Thus, the nonspecific binding property of PV1 lies in the heavy chain constant region of this particular antibody and it is eliminated upon chimerization.

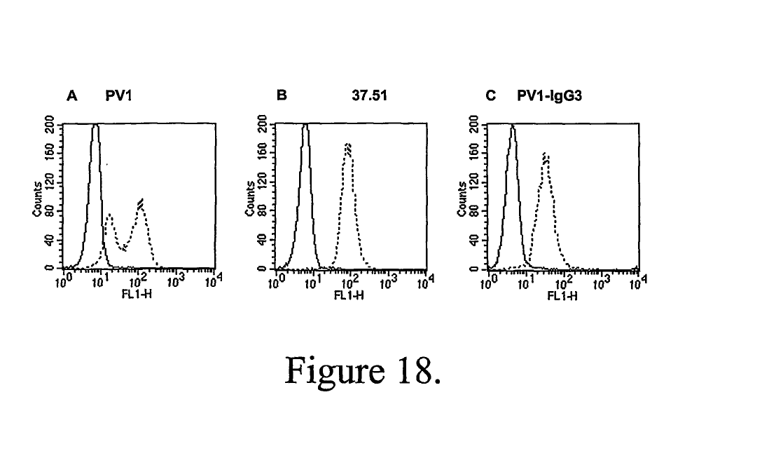

To demonstrate that PV1-IgG3 contains the CD28-specific binding activity, we used the FACScan competition assay. In these experiments, R-PE-conjugated PV1 was mixed with excess (25-fold) unlabeled PV1, PV1-IgG3 or mouse IgG3 control, and the mixture was used to stain EL4 cells. As shown in FIG. 18A, both PV1 and PV1-IgG3, but not isotype control, prevented R-PE-conjugated PV1 from binding to EL4 cells. The inhibition by PV1-IgG3 was less than that by PV1, and we interpreted these data as PV1-IgG3 competed with R-PE-conjugated PV1 for the CD28 sites but not for the nonspecific sites. Similarly, both 145.2C11(Armenian hamster anti-murine CD3) and the chimeric 145.2C11-IgG3 prevented R-PE-conjugated 145.2C11 from binding to EL4 cells (FIG. 18B), but the chimeric antibody is less efficient due to its inability to eliminate R-PE-145.2C11's nonspecific binding to cells.

We also did the reverse competition experiment using excess (25-fold) PV1 to compete with PV1-IgG3 for binding to EL4 cells. Although PV-1-IgG3 was not labeled in this case, it was specifically recognized by the FITC-conjugated donkey anti-mouse antibodies. The results in FIG. 18C showed that the inhibition of PV1-IgG3's binding to EL4 cells by excess PV1 was almost complete, demonstrating that PV1 and PV1-IgG3 bind to the same epitope.

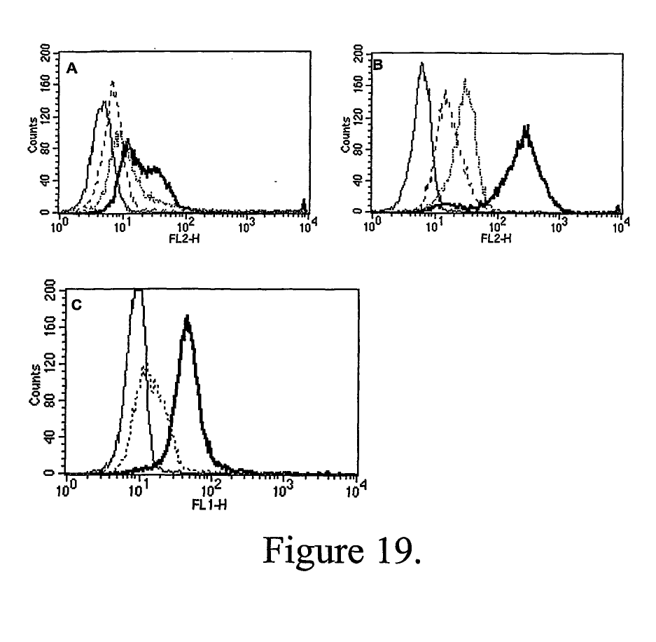

Finally, PV1-IgG3 was used to stain mouse splenic cells. PV1-IgG3-coated splenic cells were specifically recognized by the secondary antibodies R-PE-conjugated goat anti-mouse IgG3. Simultaneously, FITC-conjugated 145.2C11 was also added to splenic cells to label CD3-positive cells. In the two-color flow cytometry analysis. PV1-IgG3 specifically stained CD3-positive cells, but not CD3-negative cells (FIG. 19B). Mouse IgG3 isotype control, on the other hand, did not stain the CD3-positive cells (FIG. 19A). Thus, the chimeric PV1-IgG3 recognizes an antigen that is expressed on murine T cells, an antigen binding activity that is consistent with an anti-CD28 antibody.

### Example 19

### Induction of Collagen Induced Arthritis

Methods

Mice were immunized intradermally at the base of the tail with 125 μg of bovine CII (Collagen Gijutsu Kenkyukai, Japan) emulsified with an equal volume of CFA (Wako, Japan). Mice were boosted by intradermal injection with 125 μg of bovine CII in CFA on day 21. Mice were treated anti-CD28 antibody (PV1-IgG3) at the dose of 1 mg/kg/day continuous infusion via osmotic pump for 7 days after the initial immunization. Arthritis development was checked by inspection of four paws on day 11 after the second immunization, and the inflammation of four paws was graded from 0 to 3 as described previously (Tada, Y., A. Ho, D.-R. Koh, T. W. Mak. 1996. J. Immunol. 156:4520,. Tada, Y., A. Ho, T. Matsuyama, T. W. Mak. 1997. J. Exp. Med. 185:231). Each paw was graded and the four scores were added such that the maximal score per mouse was 12. The arthritis index was calculated by dividing the total score of the experimental mice by the number of the total number of mice.

Results

Mice were immunized with bovine CII, and observed for development of arthritis. At day 11 after the second immunization, arthritis index was significantly reduced in mice treated with anti-CD28 antibody (0.63±0.50) (P<0.01) versus control (7.50±0.66).

### Example 20

Methods

Mice;Animals

Female BALB/c and C3H mice were obtained from Charles River Japan, Inc. (Yokohama, Japan). Animals were all housed in a specific pathogen-free facility in microisolator cages with filtered air and free access to food and water. All mice were 6-8 wk of age when experiments were initiated.

Antibodies;

Anti-mouse silent CD28 (PV1-IgG3) has identical specificity to that of PV-1 clone but it does not have strong agonistic activity in vitro (Fc→IgG3):Anti-mouse CD154 (TRAP1, IgG1) was purchased from BD PharMingen (San Diego, Calif.). CTLA4-Ig (CTLA-4/Fc Chinera) was purchased from Genzyme (Cambridge, Mass.).

Tail-Skin Transplantation;

Full thickness skin grafts (0.5 cm2) from tail of donor mice(BALB/c:H-2d) were transplanted on the dorsal thorax of recipient mice(C3H:H-2b) and secured with a band-aid for 7 days. Graft survival was then followed by daily visual inspection Rejection was defined as the >80% loss of viable epidermal graft tissue. Statistical analyses were performed using a Dunnett's Multiple Comparison test. Values of p<0.05 were considered significant.

Treatment Protocols;

Skin graft recipients were treated with 10, 50, 250 μg of anti-mouse silent CD28, 250 μg of anti-mouse CD154 and 100 μg of CTLA4-Ig administered i.p. on the day of transplantation (day 0) and on postoperative days 3, and 6.

Results;

Simultaneous blockade of the CD40 and CD28 T cell costimulatory pathways by administration of anti-mouse silent CD28 and anti-mouse CD154 effectively promotes skin allograft survival in C3H mice. Control animals rejected their grafts at 9 days. Anti-CD40L mAb alone modestly prolonged allograft survival (MST 10 days), but was seen to dramatically improve survival when combined with CD28 extending median survival time-(MST-) beyond 33 days. This strategy is markedly less effective in administration of CTLA4-Ig and anti-mouse CD40L mAb, with MST of 12 days.

### Example 21

### Preparation of Fab and F(ab′)2 Fragment and of Anti-CD28 Antibody

Preparation of Fab Fragment of Anti-CD28 Antibody

Anti-human CD28 antibody (HuTN228) was digested with immobilized-Ficin (Pierce, USA). Immobilized ficin was activated with 50 mM Tris-HCL pH 6.8 buffer containing 5 mM EDTA and 11.5 mM cysteine.HCl and packed to a column. Antibody solution was added to the column, and incubated at 37° C. for 2 or 3 days. The column was washed with PBS and the digest was concentrated by ultrafiltration. The concentrated digest was applied to the gel-filtration column (TSKgel-3000SWxl, Tosoh, Japan) and appropriate fractions were collected and concentrated by ultrafiltration. Protein concentration was determined by absorbance at 280 nm (Abs280=1.4 for 1 mg/mL) and the fragment size was confirmed by SDS-PAGE.

Preparation of F(ab′)2 Fragment of Anti-CD28 Antibody

Anti-human CD28 antibody was prepared with the same method as that of Fab fragment except for the concentration of cysteine (1.15 mM) and the period of incubation (one over night).

Obviously, numerous modifications and variations of the present invention are possible in light of the above teachings. It is therefore to be understood that within the scope of the appended claims, the invention may be practiced otherwise than as specifically described herein.

All publications, patent applications, patents, and other references mentioned herein are incorporated by reference in their entirety.

What is claimed is:

1. A silenced anti-CD28 antibody.

2. The antibody of claim 1 which is a chimeric antibody.

3. The antibody of claim 1 which is a humanized antibody.

4. The antibody of claim 1 which has a variable region comprising the amino acid sequence in SEQ ID NO:2 or SEQ ID NO:4.

5. The antibody of claim 1 which has a variable region comprising the amino acid sequence in SEQ ID NO:2 and SEQ ID NO:4.

6. The antibody of claim 1 which has a variable region comprising the amino acid sequence in SEQ ID NO:6 or SEQ ID NO:8.

7. The antibody of claim 1 which has a variable region comprising the amino acid sequence in SEQ ID NO:6 and SEQ ID NO:8.

8. A polynucleotide encoding the antibody of claim 1.

9. The polynucleotide of claim 8 which comprises at least one polynucleotide selected from the group consisting of SEQ ID NO:1, SEQ ID NO:2, SEQ ID NO:3, and SEQ ID NO:4.

10. An expression vector comprising the polynucleotide of claim 8.

11. A host cell comprising the polynucleotide of claim 8.

12. A host cell comprising the expression vector of claim 10.

13. A method of producing a silenced anti-CD28 antibody comprising
culturing the host cell of claim 11 under conditions suitable for expression of the antibody and recovering the expressed antibody from said culture.

14. A method of producing a silenced anti-CD28 antibody comprising
culturing the host cell of claim 12 under conditions suitable for expression of the antibody and recovering the expressed antibody from said culture.

15. A method of producing a silenced anti-CD28 antibody comprising
introducing the polynucleotide of claim 8 into a host cell;
culturing the host cell under conditions suitable for expression of the antibody, and recovering the expressed antibody from said culture.

16. The method of claim 15 wherein said polynucleotide comprises at least one polynucleotide selected from the group consisting of SEQ ID NO:1, SEQ ID NO:2, SEQ ID NO:3, and SEQ ID NO:4.

17. A method of producing a silenced anti-CD28 antibody comprising
introducing the expression vector of claim 10 into a host cell;
culturing the host cell under conditions suitable for expression of the antibody; and recovering the expressed antibody from said culture.

18. A pharmaceutical composition comprising the silenced anti-CD28 antibody of claim 1 and a pharmaceutically acceptable ingredient.

19. A method of inducing T-cell tolerance in a patient comprising administering an effective amount of the antibody of claim 1 t to induce T-cell tolerance to said patient.

20. The method of claim 19, wherein said administering further comprises administering another immunosuppressant.

21. A method of providing immunosuppression in a patient comprising administering an effective amount of the antibody of claim 1 to provide immunosuppression to said patient.

22. The method of claim 21, wherein said administering further comprises administering another immunosuppressant.

23. A method of treating organ or tissue transplant rejection in a patient comprising administering an effective amount of the antibody to treat organ or tissue transplant rejection in said patient.

24. The method of claim 23, wherein said administering further comprises administering another immunosuppressant.

25. An antibody selected from the group consisting of HuTN228 and MuTN228 and Fab fragments thereof and F(ab)′2 fragments thereof.
